(19) United States
(12) Patent Application Publication
(10) Pub. No.: US 2021/0285004 A1
(43) Pub. Date: Sep. 16, 2021

(54) Bidirectional Constitutive Promoter and Uses, and Transfection Methods for B. Microti

(71) Applicant: MJKS Research LLC, Highland Park, NJ (US)

(72) Inventors: Kavitha Govindasamy, Edison, NJ (US); Agam P. Singh, New Delhi (IN); Dabbu Kumar Jaijyan, Delhi (IN)

(73) Assignee: MJKS Research LLC, Highland Park, NJ (US)

(21) Appl. No.: 16/813,940

(22) Filed: Mar. 10, 2020

(51) Int. Cl. C12N 15/65; C12Q 1/6897; C07K 14/435; C12Q 1/6893

(52) U.S. Cl. CPC C12N 15/65; C12Q 1/6897; C12N 2810/65; C12Q 1/6893; C07K 14/43595

(57) ABSTRACT

The present invention is directed to a novel B. microti-based promoter and expression system. The present invention is also directed to a novel method of transfecting B. microti by electroporation, the resulting transfected B. microti parasite cell lines, and optionally, B. microti transfected with a particular novel promoter. In some embodiments, transgenic B. microti parasites that express reporter genes and their uses are provided. In certain embodiments, a multi-functional promoter controls multiple genes, e.g. a bifunctional promoter may control a gene of interest and a reporter or selection gene. The promoter may be particularly useful to control expression of apicomplexan genes and form apicomplexan proteins, e.g., without limitation, those of Babesia parasites. In embodiments, expression systems comprising the promoter are provided. In certain embodiments, B. microti-based systems that express reporter genes are provided. Other expression platforms include surrogate and non-surrogate systems, transgenic parasites, bacterial, fungal, algae, and mammalian platforms, among others. These and other embodiments may be advantageously used for protein expression, diagnostic tools, disease diagnosis, identification of novel genes for drug discovery and vaccine development, recombinant antigens and other immunogens for vaccination, improved vaccine production, gene therapy, analysis of parasite proteins including structure-function analysis, candidate drug screening and profiling, and many related applications. Transfected B. microti parasites expressing reporter genes may advantageously be used e.g. in the study of the B. microti life cycle and host-parasite interaction analysis.

### REFERENCE TO A SEQUENCE LISTING SUBMITTED VIA EFS-WEB

The content of the ASCII text file of the sequence listing named “SEQPROTOCOL-16688-002-R7_ST25.txt” which is 24,281 bytes in size was created on Mar. 6, 2020 and is electronically submitted herewith is incorporated herein by reference in its entirety.

### FIELD OF THE INVENTION

The present invention is directed to a novel B. microti-based promoter and expression system. The present invention is also directed to a novel method of transfecting B. microti by electroporation, the resulting transfected B. microti parasite cell lines, and optionally, B. microti transfected with a particular novel promoter. In some embodiments, transgenic B. microti parasites that express reporter genes and their uses are provided. In certain embodiments, a multi-functional promoter controls multiple genes, e.g. a bifunctional promoter may control a gene of interest and a reporter or selection gene. The promoter may be particularly useful to control expression of apicomplexan genes and form apicomplexan proteins, e.g., without limitation, those of Babesia parasites. In embodiments, expression systems comprising the promoter are provided. In certain embodiments, B. microti-based systems that express reporter genes are provided. Other expression platforms include surrogate and non-surrogate systems, transgenic parasites, bacterial, fungal, algae, and mammalian platforms, among others. These and other embodiments may be advantageously used for protein expression, diagnostic tools, disease diagnosis, identification of novel genes for drug discovery and vaccine development, recombinant antigens and other immunogens for vaccination, improved vaccine production, gene therapy, analysis of parasite proteins including structure-function analysis, candidate drug screening and profiling, and many related applications. Transfected B. microti parasites expressing reporter genes may advantageously be used e.g. in the study of the B. microti life cycle and host-parasite interaction analysis.

### BACKGROUND OF THE INVENTION

Babesia microti is primarily transmitted through a bite of an infected tick and blood transfusion to various groups of animals, including many mammals such as humans.

Human babesiosis was first identified in 1957 and is caused by B. microti, an emerging apicomplexan parasite. Babesia microti is primarily transmitted through a bite of an infected tick and blood transfusion. Human babesiosis results in 6% to 9% of fatality and up to 20% in immunocompromised or elderly individuals, putting a huge economic burden on the human population. Recently, B. microti has been shown to be congenitally transmitted from an infected mother to a developing fetus which resulted in severe developmental defects in growing fetus and newborn. Splenectomized and immunocompromised patients can have severe consequences following a Babesia microti infection that could cause even death of the infected person. Babesia microti is a major cause of Red Blood Cell (RBC)-transfusion-transmitted infection, and in most cases, leads to death according to the U.S. Food and Drug Administration (FDA). Furthermore, B. microti poses a significant risk to the safety of the blood supply because asymptomatic blood donors can transmit Babesia microti to the blood recipients. Accordingly, the Babesia infection transmitted through blood or blood products stands as a major challenge for hemotherapy.

The protozoan parasite of the genus Babesia has both veterinary and human medical importance and affects a wide range of domestic and wild animals and humans. Likely owing to higher medical awareness the number of reported cases in humans is rising. These tick-borne parasites are thus increasingly considered a threat to animal and public health. Parasitic inclusions in erythrocytes (i.e. red blood cells) of cattle in Romania by Victor Babes at the end of the 19th century lead to the discovery of Babesia. These intra-erythrocytic parasites infect RBC (Red Blood Cells) and cause a hemolytic disease known as Babesiosis (human babesiosis by B. microti). Transmitted by ixodid ticks, host infection leads to host-mediated pathology and erythrocyte lysis, resulting in anemia, hyperbilirubinuria, hemoglobinuria, and possibly organ failure. Other names in use for Babesiosis include: Prioplasmosis, Texas Cattle Fever, Redwater Fever, Tick Fever, and Nantucket Fever, and others. There are over 100 species of Babesia identified and many have been reported to infect humans. Babesia belong to a larger group of parasites, the apicomplexan parasites, also known as sporozoan parasites, which include many parasites that cause diseases such as malaria. Once infected by Babesia, patients suffer from malaria-like symptoms. As a result, malaria is a common misdiagnosis for the disease.

The three most predominant species infecting humans are B. microti, B. duncani, and B. divergens. While initial cases were associated with splenectomy or other immuno-compromising conditions, immuno-competent persons infected with Babesia and not exhibiting clinical symptoms have been described, and serological surveys suggest that the infection may be under diagnosed. The largest focus of human infections in the U.S. has been along the northeastern costal region, giving rise to the name Nantucket fever, and the upper Midwest. Infection in Europe is apparently rarer than in the U.S., but more fatal. Most infections have been associated with individuals who have frequent contact with cattle. In the U.S., Babesia microti is the most common strain associated with humans, with other species infecting cattle, deer, goats, sheep and other livestock and domestic animals.

Currently there are no clearly effective drugs against babesiosis and many other hemolytic diseases. Some drugs have been shown to suppress, but not eliminate, parasitemia (parasite load). There remains a need to discover effective or improved drugs and vaccines, and as a first step, efficient systems and platforms to facilitate such and related discoveries, e.g. the identification of drug targets in Babesia and other apicomplexan parasites.

Similarly, characterization of genes of interest identified by genome mining is hampered in particular by a lack of suitable expression systems for genes and the resulting proteins they encode. An example is Plasmodium falciparum which causes malaria, for which lack of an effective vaccine and rapid emergence of drug-resistant strains make intervention attempts very challenging. Thus there is a need for the development of innovative alternative and improved systems for the expression of recombinant proteins from apicomplexan parasites.

For various parasites including Babesia, e.g. B. microti, and the related diseases cause by these, research is hampered by the lack of a tool to produce the relevant parasites. For example, a B. microti parasite that expresses easily detectable markers is not currently available.

The increasing availability of complete genome sequences for parasites, including apicomplexan parasites, along with the discovery of genes that may constitute potential targets to address related diseases, e.g. for therapy and/or vaccination, has great potential to fight disease but requires novel protein expression systems and platforms.

However, currently available and emerging expression systems suffer from various disadvantages, including lack of a compatible promoter, insufficient promoter strength, lack of an efficient method of transfection, and/or difficulties identifying successfully transfected organisms. This is a problem in particular for certain parasites including e.g. B. microti.

These and other shortcomings hamper the process of genetic manipulation and generation of genetically attenuated parasites and the development of transgenic parasite models that could enable the study of the parasite and of its life cycle. This is a problem in particular for certain parasites including e.g. B. microti.

For many apicomplexan parasites of medical interest, there are no known promoters, nor have promoters from other apicomplexans be used effectively. Also, transgenic parasites expressing reporter genes and systems to transfect these parasites are often lacking. This is a particular problem for B. microti, for which no promoter had previously been identified or described, nor are parasites transfected with a reporter gene available for B. microti. Furthermore, in contrast to other Babesia species, B. microti is expected to be particularly difficult to transfect based on the structure and inaccessibility of its nucleus, and successful transfections have not yet been reported (unlike, e.g. B. gibsoni, B. bigemina and B. bovis).

Based on a genome-wide phylogenetic analysis it has been suggested that the B. microti parasite may represent a new separate clade within the apicomplexan parasite phylogeny. The B. microti parasite is known to be significantly distant from other Babesia and apicomplexan parasites, and in vivo methods of transfection adapted to B. microti have not yet been reported.

B. microti may be especially challenging to transfect because it has the smallest nucleus among all the apicomplexan parasites, and its small nucleus size can impede successful transfection. It is thus unclear whether previously established methods of transfection for other species of Babesia or other apicomplexan parasite can be successfully adapted, especially for in vivo purposes.

In vivo cultures of parasites add a particular complication in that the parasites are consistently under host immune pressure and subsequently the parasites are cleared from the host when parasitemia is high. In contrast, in vitro cultivated parasites are not exposed to pressure by the immune system of a host organism.

To date, no method is known to be suitable to perform a stable transfection and long-term in vivo culture of the B. microti parasite.

Furthermore, to date, no in vitro method able to transfect B. microti wherein two genes are under the control of a single promoter is available.

These and other shortcomings hamper the discovery of new drugs and their targets, e.g. vaccine targets, and the development of genetic tools that could contribute to combatting B. microti infection in mammals including humans.

Therefore, there is a need in the art for new and/or improved expression systems or platforms, and methods employing them and their innovative features. In particular, there is a need for promoters compatible with one or more genera of apicomplexan parasites, e.g. with one or more species of Babesia parasite, for example, without limitation, with B. microti. Also there is a need for alternative promoters with the ability to sufficiently express genes and proteins in apicomplexan parasites such as Babesia parasites. Further there is a need for promoters improved in e.g. strength or functionality. Still further, there is a need for promoters with the ability to express more than one gene at a time. Yet further there is a need for promoters with the ability to express a gene of interest and a reporter gene as part at the same time in the same construct. Also there is a need for these improved promoters as applied to various expression systems or platforms, to improve such systems and platforms. Further there is a need for efficient transfection methods for parasites, including Babesia parasites, in particular including B. microti parasites. Still further there is a need for transgenic parasites expressing reporter genes, including Babesia parasites, and in particular including B. microti parasites. These and other features and advantages of the present invention will be explained and will become apparent to one skilled in the art through the summary of the invention that follows.

### SUMMARY OF THE INVENTION

Accordingly, embodiments of the present invention are directed to novel constitutive promoter sequences for control of one or more gene and expression of one or more protein. In some embodiments, the multi-functional promoter controls multiple genes, e.g. a bifunctional promoter may control a gene of interest and a reporter gene or selection marker. The promoter is particularly useful to control expression of apicomplexan genes and form apicomplexan proteins, e.g., without limitation, those of Babesia parasites. In embodiments, expression systems including vectors and platforms comprising the promoter are provided. Such embodiments include a variety of expression platforms, including surrogate and non-surrogate systems, transgenic parasites, bacterial, fungal, algae, and mammalian, among others. The promoters and its expression systems or platforms (using a B. microti-based promoter, e.g., without limitation, the BM promoter of SEQ ID No.1, referred to herein as “BM-CTQ41297”, “BM-CTQ41297 promoter”, or “BM promoter”), or a sequence homologous thereto, may be advantageously used for protein expression, diagnostic tools, recombinant antigens and other immunogens for vaccination, improved vaccine production, gene therapy, analysis of parasite proteins including structure-function analysis, candidate drug screening and profiling, and many related applications. Certain embodiments of the invention also include strains of B. microti parasites that express reporter genes and/or fluorescent markers (e.g., without limitation, GFP, mCherry, and/or bioluminescent markers, e.g. luciferase). Particular embodiments include, e.g., without limitation: B. microti expressing GFP, B. microti expressing mCherry, B. microti expressing luciferase, B. microti expressing both GFP and mCherry, Babesia microti expressing both GFP and luciferase. As will be apparent to a person of ordinary skill, other combinations of reporter genes with each other and/or with other genes of interest to be studied can be adapted according to the gene(s) being studied. These and similar embodiments which express one or more reporter gene, e.g. a fluorescent marker, downstream of any gene encoding a protein, can advantageously be used to study expression patterns both during the non-infectious and infectious cycle of a parasite, including, without limitation, expression in real-time.

According to an embodiment, provided is a heterologous expression system comprising a promoter sequence having 60% or more identity to SEQ ID NO:1 or a contiguous part thereof, wherein the contiguous part thereof has a length of 100 nucleotides or more.

According to an embodiment, provided is a heterologous expression system wherein the promoter sequence has 60% or more identity to a sequence selected from the group consisting of: SEQ ID NO:1.

According to an embodiment, provided is a heterologous expression system further comprising one or more ORF, CDS or gene operably linked to the promoter sequence, wherein one of the one or more ORF, CDS or gene is encoding for one or more of: a gene product, a reporter gene product, a selection marker gene product, an antibiotic resistance gene product or a combination thereof.

According to an embodiment, provided is a heterologous expression system wherein the ORF, CDS or gene is selected from one or more of the group consisting of: gfp (encoding for Green Fluorescent protein), rfp (encoding for Red fluorescent protein), luc (encoding for Luciferase) mCherry (encoding for mCherry protein), lacZ (beta-galactosidase), Blasticidin, Dehydrofolate reductase, and cat (encoding for Chloramphenicol acetyltransferase).

According to an embodiment, provided is a heterologous expression system wherein the promoter is bifunctional and is operably linked to two or more ORF, CDS or genes.

According to an embodiment, provided is a heterologous expression system further comprising a host cell selected from the group of apicomplexan, bacterial, fungal, yeast, algae, vertebrate, invertebrate, mammalian, bird, insect, and viral host cell.

According to an embodiment, provided is a heterologous expression system wherein the host cell is apicomplexan.

According to an embodiment, provided is a heterologous expression system wherein the system expresses one or more of: GFP, mCherry, and Luciferase.

According to an embodiment, provided is a heterologous expression system wherein the system expresses GFP and mCherry, or GFP and luciferase.

According to an embodiment, provided is a method of expressing a gene product or protein in an expression system as described herein, wherein the expression system comprises a promoter operably linked to control expression of an ORF, CDS or gene, wherein the sequence of the promoter has 60% or more identity to SEQ ID NO:1, or a contiguous part thereof, wherein the contiguous part thereof has a length of 100 nucleotides or more.

According to an embodiment, provided is a method of expressing a gene product or protein in an expression system as described herein, wherein the expression system comprises a cell free system or a homologous or heterologous cell-based system, and wherein the cell-based system is selected from the group consisting of: parasite, apicomplexan parasite, B. microti parasite, bacterial, fungal, yeast, algae, vertebrate, invertebrate, mammalian, avian, insect, viral, E. coli, S. saccharomyces, virus-infected cells, Baculovirus-infected cells, insect cells infected with a virus including baculovirus, Tetrahymena thermophila, Dictyostelium discoideum, mammalian cell line, P. pasto, Salmonella.

According to an embodiment, provided is a method of expressing a gene product or protein in an expression system as described herein, wherein the expression system further comprises one or more reporter gene selected from the group comprising: GFP, mCherry, luciferase; wherein the expression system is comprised within a parasite; and wherein the parasite is detected in a host, or a cell thereof, by a signal derived from expression of the reporter gene; wherein a host, or a cell thereof, is exposed to the parasite for sufficient duration and under conditions that allow for infection of one or more host cells by the parasite; and wherein the presence or location of the parasite in the host, or a cell thereof, is determined qualitatively or quantitatively by a signal of the reporter gene detectable by a suitable detection system, the detection system comprising: confocal microscopy, fluorescent microscopy, and an in vivo imaging system for bioluminescence monitoring.

According to an embodiment, provided is a method of expressing a gene product or protein in an expression system as described herein; wherein the expression system further comprises one or more reporter gene which provides a signal detectable by a suitable detection system; wherein signal detection is performed over sufficient time to determine parasite growth or developmental stages of the parasite, and wherein the stages comprise one or more of: gamete formation, fertilization, zygote formation, sporozoite development and the stage of parasite-infected red blood cells, the stage of parasite-infected red blood cells comprising one or more of ring stage, trophozoite stage, and gematocyte stage.

According to an embodiment, provided is a method of expressing a gene product or protein in an expression system; wherein the expression system further comprises one or more reporter gene which provides a signal detectable by a suitable detection system; wherein a parasite infected host, or a cell thereof, is further exposed to one or more drug or substance; and wherein an increased or reduced signal for the drug or substance-exposed parasite infected host, or cell thereof, in comparison to a control not exposed to the one or more drug or substance is determined.

According to an embodiment, provided is a method of stably transfecting B. microti, wherein the transfection is performed by electroporation.

According to an embodiment, provided is a B. microti parasite cell line wherein the cell line comprises a heterologous expression system, and wherein the heterologous expression system comprises one or more reporter gene, and wherein the reporter gene, or a combination of two reporter genes, is selected from the group comprising: GFP, luciferase (Luc), mCherry, GFP combined with Luc, GFP combined with mCherry, and mCherry combined with Luc.

According to an embodiment, provided is a method of expressing a gene product or protein in an expression system, wherein the cell line is a Babesia microti parasite cell line stably transfected with a heterologous expression system by electroporation, and the stably transfected B. microti parasite cell line expresses one or more reporter gene.

According to an embodiment, provided is a method with an expression system wherein the expression system further comprises one or more reporter gene selected from the group comprising: GFP, mCherry, luciferase; wherein the expression system is comprised within a parasite; and wherein the parasite is detected in a host, or a cell thereof, by a signal derived from expression of the reporter gene; wherein a host, or a cell thereof, is exposed to the parasite for sufficient duration and under conditions that allow for infection of one or more host cells by the parasite; and wherein the presence or location of the parasite in the host, or a cell thereof, is determined qualitatively or quantitatively by a signal of the reporter gene detectable by a suitable detection system, the detection system comprising an imaging system, and the imaging system selected from the group comprising: confocal microscopy, fluorescent microscopy, and an in vivo imaging system for bioluminescence monitoring.

According to an embodiment, provided is a method with an expression system as described herein, wherein detection is performed over sufficient time to determine B. microti parasite growth or developmental stages of the parasite, and wherein the stages comprise one or more of: gamete formation, fertilization, zygote formation, sporozoite development and the stage of parasite-infected red blood cells, the stage of parasite-infected red blood cells comprising one or more of ring stage, trophozoite stage, and gematocyte stage.

According to an embodiment, provided is a method with an expression system as described herein, wherein a parasite infected host, or a cell thereof, is further exposed to one or more drug or substance; and wherein an increased or reduced signal for the drug or substance-exposed parasite infected host, or cell thereof, in comparison to a control not exposed to the one or more drug or substance is determined.

According to an embodiment, provided is a method wherein B. microti or an expression system based on its promoter, e.g. a BM promoter as described herein, is cultured in vivo in a host having an immune system, and during culture is exposed to the immune system of the host.

### BRIEF DESCRIPTION OF THE DRAWINGS

FIG. 1a illustrates the genomic locus of B. microti where a B. microti-based promoter (here: SEQ ID NO:1) is located.

FIG. 1b illustrates a variety of constructs used in transient transfection of parasites (here: B. microti).

FIG. 1c illustrates expression of fluorescent genes comprised in the constructs for transient transfection shown in FIG. 1b.

FIG. 2a illustrates stable transfection of a multifunctional construct into a parasite (here: B. microti) and its multifunctional expression, in accordance with embodiments of the present invention.

FIG. 2b shows photographs of fluorescent microscopy of stably transfected cells resulting from the construct shown in FIG. 2a.

FIG. 2c illustrates successful integration of a promoter construct comprising SEQ ID NO:1 and a reporter gene in the genomic DNA of stably transfected B. microti parasites.

FIG. 3 illustrates the strong similarity in growth characteristics of wildtype and transfected parasites (here: B. microti transfected with a BM-CTQ41297 promoter of SEQ ID NO: 1 and reporter genes), in accordance with embodiments of the present invention; the transfected B. microti parasites do not have any growth defects.

FIG. 4a illustrates a construct for transfection of a Plasmodium parasite promoter.

FIG. 4b shows photographs of fluorescent microscopy of cells transfected with the construct shown in FIG. 4a, and successful gene expression in a Babesia parasite (here: B. microti).

FIG. 5 illustrates the improved strength of a promoter (here: B. microti) compared to a strong constitutive parasite promoter from Plasmodium; in accordance with embodiments of the present invention.

### DETAILED DESCRIPTION OF THE INVENTION

Successful attempts at promoter identification and transfection into a B. microti parasite have not been reported so far and attempts have shown it to be complicated and challenging. For the first time, it is shown herein that a promoter isolated from a Plasmodium species can successfully express a gene product in B. microti, compare examples. Also for the first time, a promoter is identified in B. microti, a method of transfection is established, the promoter is characterized, and is transfected to successfully express various genes in B. microti. Again for the first time, B. microti transfected parasite cell lines that express reporter genes are provided.

In embodiments, expression systems with promoters as described herein-below are provided. For example, the promoter sequence, or a contiguous part thereof, may be amplified using a suitable method, as will be apparent to the skilled person. For amplification, e.g. sequencing, PCR and/or cloning may be used. For PCR amplification, one or more primer as described herein may be used, or alternatively, a suitable primer may be chosen as will be apparent to a person of ordinary skill. The amplified promoter sequence can then be used for transfections or cloning, for example, without limitation, into a vector, e.g., without limitation, a plasmid vector, or other expression system that allows integration into a natural or artificial self-propagating or self-replicating entity such as a plasmid, genome, chromosome, artificial chromosome, YAC (Yeast Artificial Chromosome), virus, or other self-propagating entity.

In embodiments, methods may include, without limitation, stable as well as transient transfection into a cell or an organism.

In embodiments, efficient methods may include, without limitation, stable as well as transient transfection into B. microti parasites, e.g. using electroporation.

In embodiments, novel research models of transfected B. microti that express reporter genes, e.g. without limitation, GFP, mCherry, luciferase, lacZ (encoding for beta-galactosidase), Blasticidin, Dehydrofolate reductase, and cat (encoding for Chloramphenicol acetyltransferase), and their uses are provided. As will be apparent to a person of ordinary skill, a variety of known transfection techniques may be used to transfect the promoter described herein into cells of a large variety of hosts, including Babesia parasites, other parasites, bacterial, fungal and even mammalian hosts.

The promoter as described herein may have various advantages including a significantly improved strength, e.g. when compared to the known constitutive Plasmodium promoter. For example, surprisingly, it has been found that when a heterologous expression system comprising a promoter as described herein is transfected into an apicomplexan parasite, the expression system provides expression equal to or better than that of a PBANKA DHFR promoter from P. berghei of SEQ ID NO:2. Moreover the promoter as described herein allows bidirectional control of multiple open reading frames (ORF), e.g. coding sequences, CDS or genes, and thus is able to express multiple ORF, CDS or genes (and thus, after translation, proteins) at the same time. These advantages make it ideal for any number of uses that employ a strong promoter, as will be apparent to a person of ordinary skill, some of which are described in more detail herein. Both Plasmodium and Babesia belong to the phylum of apicomplexa which contains various bloodborne parasites that target Red Blood Cells (RBC) and may cause anemia, among other symptoms.

Furthermore, without wishing to be bound by theory, it is believed that the promoter as described herein provides promoter function and its contructs can provide gene expression in many cells and organisms, including both prokaryotic and eukaryotic cells.

Apicomplexan parasites (also referred to as “apicomplexans” or “parasites” herein) can infect both invertebrates and vertebrates (including mammals and birds), and infections may cause relatively benign symptoms or serious illnesses. Apicomplexans include, for example, hemolytic disease-causing parasites of the genus Babesia (Babesiosis) and Plasmodium (Malaria). Other apicomplexan genera that infect humans are all classified as are all classified as coccidia and include: Cryptosporidium, Isospora, Cyclospora, Sarcocystis and Toxoplasma. The coccidia are characterized by a thick walled oocyst stage typically excreted with the feces; some (Cryptosporidium, Cyclospora, Isospora) carry out their entire life cycle within the intestinal epithelial cells of the host and are transmitted by the fecal-oral route. Others (Sarcocystis, Toxoplasma) have complicated life cycle that involves tissue cysts and multiple hosts. The coccidia are generally considered opportunistic pathogens and often associated with AIDS. Important apicomplexans in veterinary medicine include Babesia and Theileria in cattle, and Eimeria in poultry.

Babesia and Theileria are closely related and form a group called the piroplasms, in reference to intraerythrocytic forms that are pear-shaped in some species. Babesia and Theileria exhibit a very similar life cycle, and both can infect livestock, in particular cattle, and can cause tremendous loss of livestock. The two piroplasm genera are usually distinguished by differences in their life cycles, namely a pre-erythrocytic stage exhibited by Theileria species (and generally a lack thereof in Babesia), and lack of transovarial transmission in Theileria. However, the pre-erythrocytic stage may also occur in B. microti. Babesia species can be grouped into “large” and “small” Babesia, with “small” Babesia, believed to be more closely related to Theileria based on molecular data; consistent with this none of the “small” Babesia (in contrast to the large) appears to be transmitted transovarially in ticks. Theileria species infect and cause disease in livestock, the most serious including East Coast fever of cattle caused by T. parva, with 90-100 percent mortality in some geographical areas, while T. annulata causes a milder disease of cattle.

In the pre-erythrocytic stage (e.g. present for Theileria, and also for parasites that cause malaria, such as Plasmodium falciparum, in particular for its liver stage), which occurs in the mammalian host, sporozoites invade lymphocytes and induce proliferation of the host lymphocytes. The parasite develops into a multinucleated schizont/meront which undergoes division coincident with the replication of the proliferating lymphocyte. This way the schizont is transferred to each of the daughter lymphocytes. The resulting merozoites invade erythrocytes and ultimately develop into gamonts which are infectious for the tick. The lymphoproliferative process can lead to the severe disease manifestations. Lymphocyte transformation is reversible as treatment leads to parasite clearance and subsequent lymphocyte proliferation is inhibited.

The “sporozoite stage” as used herein refers to a stage wherein the parasite is present in an insect, for example, without limitation, a tick (or other insect host). The parasite form that resides in the salivary gland of infected ticks is the sporozoite form and the stage is referred to as sporozoite stage accordingly. The sporozoites in the salivary gland of infected ticks are infectious and can produce infection when they come in contact with the blood e.g. when injected into another animal that the insect or tick feeds on during tick feeding. Blood stages are the stages of the parasite life cycle wherein the parasite is present in the blood, e.g. of the animal infected by the insect or tick. These blood stages of the parasite include the ring stage, the trophozoite stage, and the gematocyte stage, and further the tetrad stage. The ring stage is formed in the infected red blood cell (RBC) when the parasite invades the RBC. It is a very early stage that is characterized visually by the appearance of a ring-like structure when observed through a microscope. After the ring stage, the trophozoite stage follows; this is the developing stage of the parasite in the RBC. A ring develops into a trophozoite in the invaded/parasitized RBC. The parasite in the trophozoite stage is bigger and visually appears more rounded than in the ring stage. Some of the tropozoites develop into the gematocytes and some form the tetrad stage inside the RBC. As is the case for the other blood stages, the gematocyte stage is present in the blood of an infected animal (including e.g. a human); subsequently the gematocytes further develop into gametes in the insect e.g. tick, e.g. when a tick takes blood from an already infected animal.

In embodiments, mammalian hosts may include any reservoirs for Babesia species, for example, without limitation, various mammals such as deer (Cervidae), goats (Caprinae), other ruminants and rodents, roe deer (Capreolus c. capreolus), red deer (Cervus e. elaphus), alpine chamois (Rupicapra r. rupicapra), alpine ibex (Capra i. ibex).

In embodiments, a babesia species (or material derivable from such a species) detected in ruminants may be employed. Babesia spp. that have been detected in ruminants such as deer include, without limitation: B. divergens, B. capreoli, Babesia sp. EU1 (also known as B. venatorum), Babesia sp. CHI and B. motasi.

In embodiments, a babesia species (or material derivable from such a species) detected in cattle may be employed. B. divergensis, B. bigemina and B. bovis have been found in diseased cattle. Bovine babesiosis, also known as redwater, is the worldwide most important hemoparasitic diseases of cattle that causes significant morbidity and mortality.

In embodiments, the babesia species (or material derivable from such a species) may be B. divergensis which is the principal agent of babesiosis in cattle and can also infect other hosts such as gerbils (Meriones unguiculatus), sheep (Ovis aries), reindeer (Rangifer t. tarandus), and has been reported as causative agent of fatal disease in immunosuppressed or splenectomized humans.

In embodiments, the babesia species (or material derivable from such a species) may be Babesia sp. EU1 (aka B. venatorum) which has been isolated from a diseased human patient.

In embodiments, the babesia species (or material derivable from such a species) may be any Babesia species (B. spp.), for example, without limitation: B. annae, B. ardeae, B. behnkei, B. bennetti, B. bicornis, B. bigemina, B. bovis, B. caballi, B. canis, B. canis canis, B. canis rossi, B. canis vogeli, B. capreoli, B. conradae, B. crassa, B. divergens, B. duncani, B. felis, B. gibsoni, B. hongkongensis, B. kiwiensis, B. lengan, B. leo, B. lohae, B. mackerrasorum, B. major, B. microti, B. motasi, B. muratovi, B. sp. ‘North Carolina dog’, B. occultans, B. odocoilei, B. orientalis, B. ovata, B. ovis, B. pecorum, B. peircei, B. poelea, B. rodhaini, B. ugwidiensis, B. venatorum, B. vesperuginis, B. vitalii, and B. vulpes.

In embodiments, provided is the sequence of SEQ ID NO:1 or a sequence 60% or more homologous thereto and retaining promoter function in a Babesia parasite, in particular, without limitation, B. microti. Promoter function can be easily tested by conventional methods such as transfection and a reporter gene assay, as will be apparent to a person of ordinary skill, and as described herein e.g. in the examples, e.g. in example 4.

In embodiments, the promoters described herein include promoters of SEQ ID NO:1 (isolated from B. microti), or any partial consecutive sequence thereof, and may include sequences that are 60% or more identical to the sequence or a part thereof (e.g. a homologous sequence, or partially homologous sequence); such sequences may e.g. be derivable from an apicomplexan parasite, or other organisms, including host organisms (mammals, ticks, etc.). Such sequences may be identified as will be apparent to a person of ordinary skill in the art, e.g. by hybridization or in silico by sequencing, including full genome sequencing, e.g., without limitation, of a genome of an apicomplexan or babesia species as described herein, and a sequence search using SEQ ID NO:1 based on identity and/or homology. Preferably sequences may include those 55, 60, 65, 70, 75, 80, 08, 90, 91%, 92%, 93%, 94%, 95%, 96%, 97%, 98%, 99%, or more identical to SEQ ID NO:1. Sequences may also include truncated or re-arranged sequences, for example those with deletions, substitutions, insertions, and/or truncations. For example, the 3′ end of the promoter, the 5′ end of the promoter, or both, may lack up to 50, 100, 150, 200, 250 or 300, 400, 500, 600, 700, 800, 900, 1000, 1100 or more nucleotides (truncation). Alternatively or additionally, the promoter sequence between the 5′ and 3′ end may lack up to 50, 100, 150, 200, 250, 300 or more nucleotides (deletion). Other examples include sequences wherein one or more nucleotides are inserted into the sequence between the 3′ and 5′ end; and insertion may be a single nucleotide, or multiple nucleotides, or groups of three nucleotides (e.g. 3, 6, 9, 12, 15, etc.). For example, an insertion of three nucleotides may add one amino acid, after translation into a protein. Insertions may be single at one location, or multiple insertions at multiple locations.

A summary table of the sequences which includes promoter, comparative promoter sequences and illustrative constructs, gene target/recombination locus, primers and reporter genes follows below:

| SEQ | | Length |
|---|---|---|
| ID NO: | Description | (bp) |
| 1 | Promoter isolated from Babesia microti (“BM | 1969 |
| | promoter” or “BM-CTQ41297”) | |
| 2 | PbANKA DHFR-TS promoter from Plasmodium | 2262 |
| 3 | hEF1b promoter | 1601 |
| 4 | Homologous region (“arm”) 1 (HR1) for stable | 820 |
| | integration of GFP-mCherry cassette in B. microti | |
| 5 | Homologous region (“arm”) 2 (HR2) for stable | 716 |
| | integration of GFP-mCherry cassette in B. microti | |
| 6 | PBSK plasmid for construct design and cloning | 2958 |
| 7 | gDNA target locus for stable integration at 468280- | 200 |
| | 468480 of B. microti genomic sequence of Accession | |
| | number GCA_000691945.2. | |
| 8 | P1-XHO1 forward primer for PCR and cloning of the | 30 |
| | BM-CTQ41297 promoter into PBSK vector | |
| 9 | P2-HINDIII reverse primer for PCR and cloning of the | 32 |
| | BM-CTQ41297 promoter into PBSK vector | |
| 10 | P3-HINDIII GFP forward for PCR and cloning of | 33 |
| | GFP into PBSK vector | |
| 11 | P4-BAMH1 GFP reverse primer for PCR and cloning | 32 |
| | of GFP into PBSK | |
| 12 | P5-BAMH1 forward primer for PCR and cloning of | 31 |
| | 3′UTR for gene termination | |
| 13 | P6-NOT1 reverse primer for PCR and cloning of | 37 |
| | 3′UTR for gene termination | |
| 14 | P7-XBA1 primer for PCR and cloning of mCherry | 33 |
| | gene into pUC 19 vector | |
| 15 | P8 SMA1 primer for PCR and cloning of mCherry | 33 |
| | gene into pUC 19 vector | |
| 16 | P9-SMA1 primer for PCR and cloning of 3′UTR | 33 |
| 17 | P10-SMA1 primer for PCR and cloning of 3′UTR. | 32 |
| 18 | P11-XHO1 primer used for PCR and cloning of | 33 |
| | mCherry-3′UTR into pBSK vector | |
| 19 | P12-XHO1 primer used for PCR and cloning of | 32 |
| | mCherry-3′UTR into pBSK vector | |
| 20 | P13-XHO1 primer used for the PCR and cloning of | 32 |
| | 5′UTR of PBANKA DHFR | |
| 21 | P14-ECOR1 primer used for the PCR and cloning of | 29 |
| | 5′UTR of PBANKA DHFR | |
| 22 | P15-ECOR1 primer used for PCR and cloning of GFP | 33 |
| | gene into PBSK vector | |
| 23 | P16-BAMH1 primer used for PCR and cloning of | 31 |
| | GFP gene into PBSK vector | |
| 24 | P17-KPN1 primer used for PCR and cloning of HR1 | 32 |
| 25 | P18 primer used for the PCR and cloning of HR1 into | 32 |
| | PBSK vector | |
| 26 | P19 primer used for the PCR and cloning of HR2 into | 34 |
| | PBSK vector | |
| 27 | P20 primer used for the PCR and cloning of HR2 into | 32 |
| | PBSK vector | |
| 28 | P21 primer used for the diagnostic PCR for | 27 |
| | 3′integration specific PCR | |
| 29 | P22 primer used for the diagnostic PCR for | 25 |
| | 3′integration specific PCR | |
| 30 | P23 primer used for the diagnostic PCR for | 22 |
| | 5′integration specific PCR | |
| 31 | P24 primer used for the diagnostic PCR for | 26 |
| | 5′integration specific PCR | |
| 32 | GFP ORF | 717 |
| 33 | Mcherry ORF | 711 |
| 34 | Luciferase ORF | 1650 |
| 35 | 3′UTR sequence used for termination of reporter | 627 |
| | genes. | |

In embodiments, the sequence of SEQ ID NO:1 (referred to herein as “BM promoter” or “BM-CTQ41297”) or a sequence homologous thereto (referred to herein as “BM-based promoter”), may be a partial sequence; the partial sequence may have a length of 100, 150, 200, 250 or 300, 400, 500, 600, 700, 800, 900, 1000, 1100, 1200, 1300, 1400, 1500, 1600, 1700, 1800, 1900, 1950 or more nucleotides (in case of longer sequences, a part of the sequence will be identical to SEQ ID NO:1, and another part or parts, e.g. one or more insertion, will be an additional sequence). Promoter function of such partial sequences can be easily tested by conventional methods such as transfection and a reporter gene assay, as will be apparent to a person of ordinary skill, and as described herein e.g. in the examples, e.g. example 4.

In embodiments, a BM-based promoter such as the sequence of SEQ ID NO:1 or a sequence homologous thereto, may be variant sequences subject to truncation, deletion, substitution or modification etc. as described herein, yet retain its function as a promoter in one or more parasite, in particular, without limitation, in B. microti, as will be apparent to a person of ordinary skill. Such variant embodiments and their function can be easily tested for their promoter function by conventional methods such as transfection and a reporter gene assay, as will be apparent to a person of ordinary skill, and as described herein e.g. in the examples, see e.g. example 4.

In embodiments, a BM-based promoter such as the sequence of SEQ ID NO:1 or a sequence homologous thereto, may be modified (e.g., without limitation, by truncation, deletion, substitution, or addition of a partial sequence including one or more regulatory element), which may retain activity or lead to increased activity in a parasite similar to B. microti and/or a Babesia species or a parasite similar to a Babesia parasite, as will be apparent to a person of ordinary skill. Such variant embodiments and their function can be easily tested for their promoter function by conventional methods such as transfection and a reporter gene assay, as will be apparent to a person of ordinary skill, and as described herein e.g. in the examples, e.g. example 4.

In embodiments, a BM-based promoter such as the sequence of SEQ ID NO:1 or a sequence homologous thereto, may be modified as described herein-above but retain a section of SEQ ID NO:1 stretching from its bp 700 to 1300. Without wishing to be bound by theory, it is believed this section of the promoter sequence may contribute to providing promoter function, in particular, one or more of promoter strength, bidirectionality and cross-species functionality (e.g. providing promoter functionality in both prokaryotes and eukaryotes, and/or providing promoter function in parasite species other than B. microti, including Babesia species and other related parasite species as described herein).

In embodiments, a BM-based promoter such as the sequence of SEQ ID NO:1 or a sequence homologous thereto, may be modified as described herein-above but retain a section stretching from bp 778 to 1203 of SEQ ID NO:1. Without wishing to be bound by theory, it is believed this section of the promoter sequence may contribute to transcription factor binding and may contribute to providing promoter function, in particular, one or more of promoter strength, bidirectionality and cross-species functionality.

Similarly, in embodiments, a BM-based promoter such as the sequence of SEQ ID NO:1 or a sequence homologous thereto, may be modified as described herein-above, e.g. to result in an overall sequence identity of 60%, but retain, or substantially retain, e.g. at 70%, 80%, 85%, 90%, 91%, 92%, 93%, 91%, 92%, 93%, 94%, 95%, 96%, 97%, 98%, 99% or more sequence identity, one or more of the following sections of SEQ ID NO:1 shown in the table below:

| Sections of | First nucleotide | Last nucleotide |
|---|---|---|
| SEQ ID NO:1 | of section | of section |
| A | 700 | 1,300 |
| B | 778 | 1,203 |
| C | 796 | 833 |
| D | 1,166 | 1,203 |
| E | 1,698 | 1,733 |
| F | 418 | 457 |
| G | 33 | 70 |
| H | 778 | 833 |
| I | 1,152 | 1,203 |
| J | 1,678 | 1,733 |
| K | 60 | 80 |
| L | 1,122 | 1,590 |
| M | 162 | 1,432 |

Without wishing to be bound by theory, it is believed the sections of the promoter sequence in the table above may contribute to transcription factor binding and may contribute to providing promoter function, in particular, one or more of promoter strength, bidirectionality and cross-species functionality.

In embodiments, suitable BM-based promoter sequences include SEQ ID NO:1, and any sequences that are 60% or more identical to SEQ ID NO:1 and that provide promoter function in one or more apicomplexan parasite, Babesia spp. parasite, mammalian, fungi or bacterial host cell or strain, or a virus host or strain. Suitable promoter sequences may be derivable from one or more apicomplexan parasite, e.g., without limitation, a species of Babesia, e.g., without limitation, the species enumerated herein. Suitable promoter sequences may be 55, 60, 70, 75, 80, 85, 85, 90, 91%, 92%, 93% 91, 92, 93, 94, 95, 96, 97, 98, 99 or more % identical to SEQ ID NO:1. Such sequences may be identified as will be apparent to a person of ordinary skill in the art, e.g. by hybridization or in silico by sequencing, including full genome sequencing, and a sequence search using SEQ ID NO:1 based on identity and/or homology, and/or cloning the homologous promoter into a reporter vector with a reporter gene as described herein, and testing for suitable expression strength, e.g. in comparison to a particular organism and its constitutive promoter, and determining equal or better strength. Promoters having an increased expression strength of at least 1.2, 1.3, 1.4, 1.5, 1.6, 1.7, 1.8, 1.9 times or more when compared to a DHFR promoter (a strong constitutive promoter originating from Plasmodium berghei that is expressed in other apicomplexan parasites such as e.g. B. microti) are be preferred, e.g. at least 2, 3, 4, 5, 6, or more times. For example, the promoter of SEQ ID NO:1 has a strength that is about 6 times that of the DHFR promoter, as shown in example 4 herein-below.

In embodiments, the promoters described herein may have an expression strength that when compared to a constitutive DHFR promoter of Plasmodium berghei of SEQ ID NO: 2 in the same vector is equal or higher. A comparison may be performed as described herein-below in example 4.

In embodiments, the promoters described herein may be used for expression of a gene of interest in any appropriate expression system, including, e.g., without limitation, mammalian, fungi, apicomplexan parasite, bacterial or viral expression systems. Many different suitable expression systems/platforms are commonly known in the art and can be adapted by exchanging the promoter and combining it to the appropriate elements of the relevant expression system, as will be apparent to a person of ordinary skill, and as described, e.g., in M. R Green & J. Sambrook, Molecular Cloning: A Laboratory Manual, 4^{th} ed. (2012), in particular, e.g., in vol. 1, chapters 3-5 and 8; vol. 2, chapters 15-16; and vol. 3, chapters 17, 19 and 22, which hereby are incorporated herein by reference in their entirety.

In embodiments, the mammal or mammalian host cell may be selected from the group consisting of: human, rodent, mouse, rat, guinea pig, gerbil, squirrel, rabbit, dog, cat, ruminant, deer, goat, sheep, cattle, or other mammal.

In embodiments, generally, to express a gene/coding sequence of interest, e.g. to express and form a recombinant protein, an expression vector containing the gene of interest under the control of a suitable promoter as described herein is constructed. The expression vector can then be transfected/introduced into cells that recognize the promoter, in which case it can be used to express the target gene or coding sequence. Alternatively, cell-free systems with the necessary machinery for protein expression can be used.

In embodiments, a vector for expression of a coding sequence or gene of interest may generally include: a promoter, a multiple cloning site, a polyadenylation signal, and an antibiotic resistance marker. Optionally, one or more enhancer sequence may be present. Many different suitable vectors are commonly known in the art and can be adapted by exchanging the promoter and combining it to the appropriate elements of the relevant vector, as will be apparent to a person of ordinary skill.

In embodiments, generally, the promoter construct may include sequences corresponding to a 5′ untranslated region (5′ UTR). The 5′ UTR (also known as a leader sequence or leader RNA) is the region of an mRNA that is directly upstream from the initiation codon; this region helps e.g. with regulation of the translation of a transcript by differing mechanisms in viruses, prokaryotes and eukaryotes. The 3′ UTR is the section of messenger RNA (mRNA) that immediately follows the translation termination codon and is known to regulate mRNA-based processes, such as mRNA localization, mRNA stability, and translation, among others. Without wishing to be bound by theory, inclusion of one or more of the 5′UTR and 3′UTR may improve one or more of regulation of translation, mRNA stability and increase promoter strength.

In embodiments, the expression vector can be introduced into suitable host cells by a variety of well-known methods to introduce foreign DNA, including, without limitation: transfection, transduction, and transformation, e.g., without limitation, electroporation. Many suitable methods and systems are commonly known in the art and can be adapted accordingly, as will be apparent to a person of ordinary skill, and as described, e.g., in M. R Green & J. Sambrook, Molecular Cloning: A Laboratory Manual, 4^{th} ed. (2012), in particular, e.g., in vol. 1, chapters 3-5 and 8; vol. 2, chapters 15-16; and vol. 3, chapters 17, 19 and 22, which hereby are incorporated herein by reference in their entirety. A suitable pET expression system in E. coli is described e.g. in Jia B and Jeon CO, High-throughput recombinant protein expression in Escherichia coli: current status and future perspectives, Open Biol. 2016; 6(8):160196, doi:10.1098/rsob.160196, which hereby is incorporated herein by reference in its entirety.

In embodiments, the promoters, vectors and expression systems described herein can be used in a multitude of applications that require or benefit from recombinant proteins from apicomplexan parasites, their cells, their tissues, and from non-apicomplexan cells or non-apicomplexan tissues, as will be apparent to a person of ordinary skill. Illustrative non-limiting examples of these numerous applications are described below.

In embodiments, heterologous expression systems are provided which may include prokaryotic, eukaryotic or viral expression systems. For example, the expression system may comprise within a suitable vector an apicomplexan promotor as described herein, an apicomplexan gene, one or more selection markers including one or more fluorescence marker and one or more antibiotic resistance markers. The vector may be transfectable into a suitable cell, organism or cell-free expression system selected from the group comprising: E. coli, S. saccharomyces, virus-infected cells, Baculovirus-infected cells, insect cells infected with a virus, e.g. baculovirus, Tetrahymena thermophila, Dictyostelium discoideum, mammalian cell line, P. pasto, Salmonella, Plasmid DNA, cell-free system. Available heterologous expression systems, in particular those for soluble immunogenic proteins, often had limited success when used for the expression of parasite proteins; thus the promoters, vectors and systems described herein provide an important alternative or additional tool. Components of the systems enumerated above will be apparent to a person of ordinary skill, and are described in detail in publications such as, e.g., in M. R. Green & J. Sambrook, Molecular Cloning: A Laboratory Manual, 4^{th} ed (2012), in particular, e.g., in vol. 1, chapters 3-5 and 8; vol. 2, chapters 15-16; and vol. 3, chapters 17, 19 and 22, which hereby are incorporated herein by reference in their entirety. For example, for E. coli based expression systems, various growth media, methods to transform the prokaryote, multiple cloning vectors and host strains are available and well known. The same applies to numerous eukaryotic and viral expression systems.

In embodiments wherein post-translational modifications in the recombinant protein are needed (e.g., without limitation, for function or antigenicity), or if bacterial systems are not performing as expected (e.g., without limitation, due to codon usage), then expression may be performed in eukaryotic systems which are available either using a surrogate organism or cell-free system, as will be apparent to a person of ordinary skill. In either case, there is a large variety of clones, strains, culture conditions, and tags that can be used for optimization, e.g. inserting the promoter as described herein into one of the available systems, and optionally modifying the system depending on the desired protein characteristics and/or modifications, depending on the desired use, as will be apparent to a person of ordinary skill.

In embodiments, heterologous eukaryotic expression systems are provided which may include chosen depending on the desired protein to be expressed and its characteristics, and/or the aim of its analysis, as will be apparent to a person of ordinary skill. Considerations to choose a suitable system include presence or absence in the native protein, and if presence than desirability of presence in the heterologous protein formed, of one or more of: post-translational modifications, glycosylation, acylation, ability to form disulfide linkages, interaction with eukaryotic chaperones, proteolytic processing, sub-cellular compartmentalization, secretion mechanisms that avoid accumulation, toxicity, and immunogenicity.

In embodiments, generally, after selection of a gene of interest, in silico analysis may establish the presence of features such as protein domains, secretion signals, organellar targeting, and post-translational modifications, if any. These and the desired use of the recombinant protein allows the selection of a suitable cloning vector and expression system. Using the promoter described herein, the gene of interest can be directly cloned into a suitable vector to express in a homologous or a surrogate system; typically a system based on a species that can be grown in large quantities and is easily genetically manipulated. In case of lack or insufficient scale of in vitro culture of a homologous system (often the case for parasites) a heterologous system can be chosen, e.g. many bacterial systems provide excellent scale (but lack other potentially desired features such as glycosylation).

In embodiments, a gene may be processed for expression in a heterologous system, and the processed gene may be inserted into a vector for the selected organism. General steps to prepare a gene for expression in a heterologous systems include one or more of: removing predicted membrane-spanning regions, avoiding disrupting predicted secondary structural elements, respecting the boundaries of known globular domains, if any; and avoiding the inclusion of low-complexity regions or hydrophobic residues at the N- and/or C-termini, as will be apparent to a person of ordinary skill. Various bacterial and eukaryotic expression systems such as yeasts have been described, e.g. in Appl. Microbiol. Biotechnol. 2006; 72:211-222 (PMID: 16791589), and references therein, which hereby is incorporated herein by reference in its entirety.

In embodiments, a gene of interest may be expressed using a promoter or vector as described herein in a yeast expression system; especially if large-scale fermenters and high protein yields are necessary, and depending on the existing glycosylation pattern of the native protein, if any, and the desired glycosylation pattern of the resulting protein. As will be apparent to a person of ordinary skill, the N- and O-linked glycosylation patterns of the recombinant proteins may be qualitatively and quantitatively different from those in the native parasite protein, potentially resulting in inactive products. Yeast based expression systems may include various Pichia pastoris and Saccharomyces cerevisiae species of which many are available, as will be apparent to a person of ordinary skill. Non-limiting examples of recombinant proteins produced in Pichia include, e.g., PfCP-2.9 and Pvs25. Expression of parasite recombinant proteins may also be achieved in form of a high throughput version of a yeast two-hybrid assay that circumvents difficulties in expressing P. falciparum proteins in Saccharomyces cerevisiae as described, e.g., by LaCount D J, et al. A protein interaction network of the malaria parasite Plasmodium falciparum. Nature. 2005; 438:103-107, which hereby is incorporated herein by reference in its entirety.

In embodiments, a gene of interest may be expressed using a promoter or vector as described herein in an expression system based on a virus- or baculovirus-mediated expression systems in insect cells. These may be a good alternative for development of vaccines, in particular if branched multiantennary glycans are not present or not needed for the desired function in the recombinant protein. The viruses are generally suitable both as delivery systems and as adjuvants. Typically in these transient expression systems, virus-mediated synthesis of heterologous proteins in insect cells and larvae yields milligram quantities of recombinant protein, with some required post-transcriptional modifications. Insect cell culture also allows to express several genes of interest simultaneously. Various viral expression systems have been used to express apicomplexan proteins and may similarly be employed using a promoter as described herein, as will be apparent to a person of ordinary skill.

In embodiments, a gene of interest may be expressed using the promoter or vector as described herein in an expression system based on the ciliate Tetrahymena thermophile, which is characterized by high cell densities and suitable for bioreactor culture. Examples of expressed parasite proteins include the glycosylphosphatidylinositol (GPI)-anchored full-length Plasmodium CSP, demonstrating recognition of targeting and GPI anchoring signals. As will be apparent to a person of ordinary skill, the published Tetrahymena genome can be used to identify secretion and organellar signals for secretion and targeting, regulatory elements for constructing transfection vectors, etc.

In embodiments, a gene of interest may be expressed using the promoter or vector as described herein in an expression system based on the slime mold Dictyostelium discoideum. As will be apparent to a person of ordinary skill, this system allows axenic in vitro culture in synthetic media, and is suitable to produce e.g., without limitation, alternative subunit vaccines against parasitic diseases such as babesiosis or malaria, as has been shown for malaria by a Plasmodium recombinant CSP anchored to the surface of Dictyostelium, which elicits antibodies against two different regions of the target protein. Advantageously, a terminal segment, here the CSP-C-terminal segment by the Dictyostelium contact site A glycosyl-phosphatidylinositol anchor signal sequence, may allow for surface display.

In embodiments, a gene of interest may be expressed using a promoter or vector as described herein in an expression system based on mammalian cell lines, as will be apparent to a person of ordinary skill. Mammalian cell lines may typically be used for, e.g., without limitation, identification and characterization of parasite surface ligands involved in host-parasite interactions, binding assays, expression of membrane transporters, and testing of DNA-based vaccines.

In embodiments, for expression in mammalian cells, a mammalian expression vector such as, without limitation, pCDNA3.1, EGFP and CMV (e.g. a cell or cell line from human, deer, goat, sheep, cattle, dog, cat, mouse, rat, rabbit, gerbil, squirrel, and others). The coding sequence may be cloned into the mammalian expression vector by methods known in the art, then the vector may be delivered to the cells of interest by a variety of known methods, e.g., without limitation, by electroporation, or the vector may be chemically delivered, as known in the art. Inside the mammalian cells, the Babesia promoter will express the cloned gene. For example, the methods may be performed generally as described for the above promoters, replacing it with the Babesia promoter described herein. Gene expression in the mammalian cells or organisms may, e.g., be performed as described for the CMV promoter and pCDNA3.1 vector in W. Xiaa et al., Protein Expression and Purification, Vol. 45, Issue 1, 2006, p. 115-124, which hereby is incorporated herein by reference in its entirety.

In embodiments, a gene of interest may be expressed using a promoter or vector as described herein in an expression system based on homologous or heterologous parasite expression systems.

In embodiments, a gene of interest may be expressed using a promoter or vector as described herein in an expression system based on homologous parasite expression systems. Enhanced production of proteins of interest in the particular parasite may be achieved by overexpression, or by increasing the parasite numbers by culture scale-up; the resulting products have the advantage over recombinant proteins produced e.g. in bacteria as they typically more faithfully display all desirable characteristics, including e.g. immunogenicity and biological activity, more similar to the proteins directly isolated from a parasite. For such culture, some chemically defined media have become available, e.g. for continuous growth of the P. falciparum intraerythrocytic stage, which can be used in a homologous system for production of Plasmodium recombinant proteins, as will be apparent to a person of ordinary skill.

In embodiments, e.g. for parasites that cannot yet be cultured in vitro, animal models can be used, as will be apparent to a person of ordinary skill. For example, without limitation, rats or other mammals may be used to substitute ruminants for enhancing propagation of parasites, e.g., without limitation, Cryptosporidium spp. Other suitable animals may include rodents, rats, mice, guinea pigs, rabbits, other mammals, birds, vertebrates and invertebrates, as will be apparent to a person of ordinary skill.

In embodiments, a gene of interest may be expressed using a promoter or vector as described herein in an expression system based on heterologous parasite expression systems based on protozoan parasites as surrogate systems, as will be apparent to a person of ordinary skill. Various such commercial heterologous expression systems are available, for example, without limitation, a eukaryotic protein expression system based on the protozoan Leishmania tarentolae (LEXSY®, Jena Bioscience®): its eukaryotic gene expression machinery includes full glycosylation and disulfide bond formation and the system offers various inducible or constitutive vectors, and a choice to target the protein to intracellular compartments or for secretion. Thus the constitutive promoter could be replaced with the Babesia promoter; alternatively or additionally, another surrogate protozoan could be used adapting accordingly, as will be apparent to a person of ordinary skill. Examples include, e.g.: Neospora caninum (expresses genes of the phylogenetically related T. gondii), engineered Toxoplasma ts-4 mutants express the Leishmania antigen kinetoplastid membrane protein-11 and elicit a specific immune response in BALB/c mice, Trichomonas foetus expresses functional Trichomonas vaginalis AP65 adhesin, Plasmodium vivax chloroquine-resistance transmembrane protein can be expressed in both P. falciparum and Dictyostelium, and Trypanosoma cruzi (Chagas disease) can be used to screen for drugs against Crithidia fasciculata ornithine decarboxylase (for a more detailed description, see e.g. Trends Parasitol. 2010 May; 26(5): 244-254, PMID: 20189877, and references therein, which hereby is incorporated herein by reference in its entirety).

In embodiments, a gene of interest may be expressed using a promoter or vector as described herein in a phylogenetically tailored heterologous expression system. Phylogenetically related organisms tend to have a higher probability of shared structures, mechanisms, and processes required for protein function and thus should preferably be chosen when testing for expression of recombinant proteins from parasites. As a first step the closest relative to a parasite organism of interest, e.g. a Babesia parasite, for example, without limitation, B. microti, that shares the targeted structure, pathway or gene is chosen. Further selection criteria for the organism to use in a new expression system or platform include: ease of genetic manipulation, compatibility with large-scale culture, e.g. in axenic conditions, and/or compatibility with a particular culture medium, e.g. a cell-free culture medium. In a second step, a suitable cloning vector is selected, depending on the native or desired targeting/location of the protein, e.g. cytosolic, particular organelle, surface expression or secretion. Further considerations include the desired use of the recombinantly expressed protein, including, without limitation: diagnostic tool development, screening, and drug profiling, as will be apparent to a person of ordinary skill.

In embodiments, a gene of interest may be expressed using a promoter or vector as described herein in a phylogenetically tailored heterologous expression system based on an engineered pseudoparasite. Alternatively or additionally, a pseudoparasite may be engineered in an organism that expresses genes that are homologous to those of the parasite of interest. This can provide various advantages, including ensuring correct protein folding and post-translational modifications that take place in the relevant organism and thus providing a specific, effective immune response. This can provide a scalable multi-antigen vaccine delivery platform which allows to identify and select improved combinations of multiple immunogens to be expressed in their immunologically relevant conformation by the pseudoparasite, and at the same time the whole engineered organism may be used as an adjuvant. The engineered pseudoparasite displaying the selected parasite antigens can then be tested for induction of an effective immune response without other adjuvants being present. Particular genes of the organism of interest may be incorporated into a pseudoparasite platform as desired. For example, genes expressed during a particular stage of the parasite (e.g. pre-erythrocytic, intra-erythrocytic, and/or post-erythrocytic) may be selected as candidates for a vaccine and may be integrated into the pseudoparasite and tested accordingly, and may be added to the platform as desired. Babesia stages according to its life cycle may include, e.g., without limitation: sporozoite, trophozoite, merozoite, gametocyte, ookinete, oocyst stage, etc. Stage-specific genes expressed at different times in the parasite's life cycle can thus identified can be incorporated to provide a multi-gene platform expressing all necessary genes to provide an effective vaccine.

In embodiments, a gene of interest may be expressed using a promoter or vector as described herein in a cell-free protein synthesis system, as will be apparent to a person of ordinary skill in the art. Available systems include, e.g., a wheat-germ cell-free expression system which has been e.g. used to produce a functional PfDHFR-TS and malaria vaccine candidates which required no prior optimization or harmonization of the P. falciparum AT-biased codon usage yet provided soluble, highly immunogenic proteins. Such systems are suitable, e.g., for proteins including those from genes with codon bias and when post-translational modifications are not required.

In embodiments, a gene of interest from an apicomplexan parasite may be expressed within the same apicomplexan parasite species, or in another apicomplexan species, in particular, a related apicomplexan species. Apicomplexan parasites, also called sporozoan parasites, include any protozoan of the typically spore-producing phylum apicomplexa, also known as sporozoa by some authorities. Apicomplexa are a monophyletic group which formerly was part of a group called sporozoa; there have been suggestions to revert back to the name sporozoa. Defining characteristic of the apicomplexa is a group of organelles found at the apical end of the organism. This apical complex includes secretory organelles known as micronemes and rhoptries, polar rings composed of microtubules, and in some species a conoid which lies within the polar rings. At some point during their life cycle, members of the apicomplexa either invade or attach to host cells. It is during this invasive (and/or motile) stage that these apical organelles are expressed. The apical organelles play a role in parasite-host cell interaction and subsequent invasion of the host cell. All apicomplexans are parasitic and lack contractile vacuoles and locomotor processes.

In embodiments, a gene of interest may be expressed using a promoter or vector as described herein in an expression system wherein a related apicomplexan species is selected based on phylogeny and/or systematics (reflecting evolutionary relationships between organisms), to select and then test species with a higher probability to produce the heterologous recombinant proteins successfully and with all desired characteristics. Similar structures and mechanisms present in distinct organisms can indicate unknown shared processes required for production of functional proteins, thus the identification of close relatives to the parasites of interest allows developing of alternative systems and platforms for expressing recombinant proteins from apicomplexan parasites. For example, an organism may be selected from within the apicomplexan group, e.g. an apicomplexan species or genus that is closely related to the one of interest, e.g. a species genetically, phylogenetically, systematically or functionally close to a species within the group of piroplasms, a species within the genus of Babesia, a species within the group of Theileria, a species within the genus of Plasmodium, B. microti, B. duncani, B. divergens, T. panva, T. annulata, Leishmania tarentolae. Other suitable babesia species may include, without limitation: B. annae, B. ardeae, B. behnkei, B. bennetti, B. bicornis, B. bigemina, B. bovis, B. caballi, B. canis, B. canis canis, B. canis rovsi, B. canis iogeli, B. capreoli, B. conradae, B. crassa, B. divergens, B. duncani, B. felis, B. gibsoni, B. hongkongensis, B. kiwiensis, B. lengau, B. leo, B. lohae, B. mackerrasorum, B. major, B. microti, B. motasi, B. muratovi, B. sp. ‘North Carolina dog’, B. occultans, B. odocoilei, B. orientalis, B. ovata, B. ovis, B. pecorum, B. peircei, B. poelea, B. rodhaini, B. ugwidiensis, B. venatorum, B. vesperuginis, B. vitalii, and B. vulpes.

Alternatively, in embodiments, a gene of interest may be expressed using a promoter or vector as described herein in an expression system wherein the related organism used is a non-apicomplexan species closely related to the apicomplexan species of interest in one or more characteristic, including genetical, phylogenetical, morphological, structural, functional, biochemical. For example, the species may include, without limitation: Perkinsus marinus, Chromera velia, other Perkinus or Chromera species, and other genera related to the apicomplexans.

Thus, in embodiments, a gene of interest may be expressed using a promoter or vector as described herein in an expression system wherein the related organism used is a non-apicomplexan species and wherein the species is a Perkinus species, including, without limitation, Perkinsus marinus. P. marinus is non-pathogenic to humans, its genome has been fully sequenced, and a transfection methodology has been established, and can be modified as necessary, as will be apparent to a person of ordinary skill. P. marinus is a protozoan parasite of mollusks within the dinoflagellate lineage that is phylogenetically close to the divergence point from the apicomplexans. Its characteristics also include structural proximity, as Perkinsus and Apicomplexa share multiple morphological features. For P. marinus, cell-free, fully defined media formulations for its high-density easily scalable culture are available thus allowing production of recombinant proteins from apicomplexan parasites that can meet qualitative and quantitative requirements for various genes/proteins of interest.

Thus, in embodiments, a gene of interest may be expressed using the Babesia promoter described herein in an expression system wherein the related organism used is a non-apicomplexan species and wherein the species is a Chromera species, including, without limitation, Chromera velia which is an algae. Chromera velia has been successfully applied where plastids are involved, and the methodology can be adjusted as necessary, as will be apparent to a person of ordinary skill. Notably, the dinoflagellates and apicomplexan plastids may have a single endosymbiotic origin and both derive from a red algal endosymbiont. Chromera can be easily grown in culture, and may be particularly suitable for successful expression of a large variety of apicomplexan plastid-encoded proteins, as well as for high-throughput screening for drugs targeting the plastid, after appropriate characterization and optimization, as will be apparent to a person of ordinary skill. The use of bioengineered algae for protein expression may be performed, e.g., generally as described in Crit. Rev. Microbiol. 2008; 34:77-88 (PMWD: 18568862), which hereby is incorporated herein by reference in its entirety.

In embodiments, a gene of interest may be expressed using a promoter or vector as described herein in an expression system wherein the existing promoter is replaced with a promoter as described herein, and after addition or replacement of any desirable elements such as 3′UTR and/or 5′UTR, selection markers, fluorescence markers, signal sequences etc. Such systems may include systems currently commercially available, or systems similar thereto using a related organism and similar vectors and methodology. In illustrative list includes, without limitation: Pichia Expression Kit® (Invitrogen®, Carlsbad, Calif.), Bac-to-Bac® baculovirus expression system (Invitrogen®, Carlsbad, Calif.), FlashBAC™ (Oxford Expression Technologies®, UK), InsertDirect™ (BioSciences Inc., USA), HEK 293FreeStyle™ (Invitrogen®, Carlsbad, Calif.), LEXSY® (Jena Bioscience®, Jena, Germany; protein expression system using Leishmania tarentolae), pET expression system (Novagen, Madison, Wis.), WREPO® (Cell Free Sciences®, Ehime, Japan; a wheat germ cell-free protein expression system). Illustrative non-commercial systems are available from the labs that have developed them, and include, without limitation: T. thermophila (Nat. Biotechnol. 1999; 17:462-465, PMID: 10331805, hereby incorporated herein by reference in its entirety), Dictyostelium (FASEB J. 2008; 22:4055-4066, PMID: 18714070, which is available at the Dictyostelium consortium website of the NIH and is hereby incorporated herein by reference in its entirety), P. marinus (Parasite (1997); 4:67-73, as e.g. published online at https://doi.org/10.1051/parasite/1997041067, hereby incorporated herein by reference in its entirety), Chromera (Nature 2008; 451:959-963, hereby incorporated herein by reference in its entirety); many of these and other organisms are publicly available via the website of the American Type Tissue Culture (ATCC); each of the aforementioned references describing such systems in additional detail is hereby incorporated herein by reference in its entirety.

Numerous methodologies, applications and uses described herein will benefit from an improved and/or increased production of recombinant proteins. The amount of protein required for extensive screenings often is too high for existing methods, especially in high-throughput methodologies which continue to require protein quantities virtually unattainable from parasites isolated from the host.

In embodiments, the promoters, vectors and expression systems as described herein may be used together with a selection marker, in particular fluorescent selection markers such as gfp (the sequence e.g. as described in U.S. Pat. No. 6,146,826 incorporated herein by reference in its entirety), mCherry, luciferin and others, as described herein, and after transfection, successfully transfected cells may be sorted/isolated using the relevant fluorescent selection marker, e.g. by Fluorescence activated cell sorting (FACS), as will be apparent to a person of ordinary skill.

In embodiments, the promoters, vectors and expression systems as described herein may be used in the development of diagnostic tools. Rapid and accurate diagnosis allows effective disease management while avoiding unnecessary use of therapeutic agents and development of drug-resistant strains. Diagnostic tool development requires substantial amounts of the parasite at particular life cycle stages, or particular proteins expressed at those stages. For such proteins, the recombinant proteins should accurately represent the native equivalents, as will be apparent to a person of ordinary skill. For example, HRP-2 and aldolase antigens have been used. Diagnostics for trypanosomiasis, babesiosis, and leishmaniasis also rely on recombinant antigens, for example as described in Hunfeld K P, et al., Babesiosis: recent insights into an ancient disease, Int. J. Parasitol. 2008; 38:1219-1237, which hereby is incorporated herein by reference in its entirety.

In embodiments, the promoters, vectors and expression systems as described herein may be used in the development of a screen of blood or blood products, including e.g. blood reserves, blood donations, and any cadaver-derived products such organs, bone or tissues for transplant use.

In embodiments, the promoters, vectors and expression systems as described herein may be used in the development of immunogens for vaccination to prophylactically treat the disease. While use of proteins isolated directly from parasites may be advantageous over recombinant proteins in that all structural and immunogenic characteristics that are native to the organism are displayed in the vaccine, their availability at the required purity standard and quantity can pose a limiting factor. In addition, polymorphism of the protein of interest can result in vaccines with poor reproducibility. In contrast, subunit vaccines typically rely on the industrial production of a recombinant antigen of choice and have low inherent safety risks associated with their manufacture processes. Systems that may be used for producing vaccine candidates include, without limitation: E. coli, S. cerevisiae, insect cells, mammalian cells, P. pasto, Salmonella, Plasmid DNA, Baculovirus, and others. Eucaryotic systems have the advantage of providing a glycosylated product which may be desirable, especially if immunogenicity is affected.

In embodiments, the protein targets that can be used e.g. as antigens for vaccination or for vaccine production may be selected as will be apparent to a person of ordinary skill. Typically a protein expressed on the cell surface and thus accessible to an antibody will be chosen. Other proteins of interest may include enzymes. For example, suitable proteins may include, without limitation: acidic basic repeat antigen (bABRA); MSP1, AMA-1 (apical membrane antigen 1); LAS-1, RTSS/ASO2A, Eimeria tenella microneme protein 2 (EtMIC2); tachyzoite-dense granules (GRA); heat-shock protein HSP; P. falciparum surface 25 (Pfs25); and P. vivax surface 25 (Pvs25), CSP, SAG1, SAG2, OP2, P23, CP15, p67, NcSAG1, NcSAG2, NcHSP33, P. falciparum liver-stage antigen 1 (LSA-1) for a pre-erythrocyte-stage protein-based vaccine, and (for Babiosis vaccines:) 12D3, 11C5, Bd37; dihydrofolate reductase-thymidylate synthase (DHFR-TS) from Babesia gibsoni; or any homologuous proteins thereof.

In embodiments, the promoters, vectors and expression systems as described herein may be used in structure-function analysis of proteins, e.g. to provide or identify essential proteins, e.g., essential parasite enzymes. Analysis of protein function may require biochemical, biophysical, and functional characterization of the protein of interest which is particularly challenging when analyzing apicomplexan parasites because there is a lack of in vitro culture methods for these obligate intracellular parasites. Thus apicomplexan promoters can be a valuable tool to analyze protein function.

In embodiments, the promoters, vectors and expression systems as described herein may be used in the development of candidate drug screening and profiling to identify and characterize drugs that are able to e.g. protect against or treat an infection with an apicomplexan parasite or one or more of its associated symptoms or complications. For example, drugs against apicomplexan parasites may be based on interference with metabolic pathways associated with the apicoplast (a relict, nonphotosynthetic plastid found in most apicomplexans, including e.g. Plasmodium falciparum). Cultured parasites often do not provide enough material for drug screening, e.g. of extensive combinatorial chemical libraries, as current drug-discovery screens require large amounts of proteins. Thus for such screening, suitable expression systems as described herein are an important tool. Various screening methodologies for candidate drugs are available, as will be apparent to a person of ordinary skill.

In embodiments, the promoters, vectors and expression systems as described herein can be used for gene therapy (e.g. by transplanting transfected cells that express a desired gene/produce a desired protein in situ in a patient), in particular to treat diseases that can be improved by strong expression of a particular gene, such as the gene coding for insulin. For example, diabetes mellitus type 1 or 2 which result from insulin deficiency may be treated by introducing to a patient's cells in situ or ex vivo a vector or construct comprising the insulin gene under control of the Babesia promoter. In case of an ex vivo procedure, the cells comprising the Babesia promoter construct are then transplanted or otherwise introduced into the human patient. Alternatively or additionally, disease treatment, e.g., without limitation, of diabetes, may be performed using gene therapy.

For gene therapy, for example, without limitation, of diabetes, generally, a suitable vector may be introduced into suitable cells which either are present in or can be transplanted into a patient who lacks sufficient expression of the insulin gene. These cells may be foreign cells or may be harvested from the patient and then re-transplanted. A suitable vector can be chosen as will be apparent to a person of ordinary skill, exchanging the promoter for a promoter as described herein, operably linked to the coding sequence of a gene of interest, here the insulin gene, as will be apparent to a person of ordinary skill. For example, a construct as described herein is suitable after replacing one of the reporter genes with the coding sequence for the insulin gene. Alternatively or additionally, a vector, e.g. a plasmid, can be introduced directly, e.g. via intramuscular or subcutaneous injection into the diseased organisms or patients. The introduced vector will express the target gene into the target protein to function at the target site and thus cure the disease. The target protein thus expressed may also be adapted to provide an improved therapy or cure, e.g. by outperforming a nonfunctional or misfolded protein; such adaptations may be structural or regulatory (e.g. the protein and thus its gene sequence may be altered to provide a different folding or increased activity, or similarly a regulatory element may be changed, added or removed).

Ixodes scapularis is the most important vector of babesiosis in the Eastern and Midwestern United States. In the United States, at least 11 species of ticks have been recognized as the vector for various pathogens. Ticks are the unique vector of many important diseases like babesiosis, Lyme borreliosis, anaplasmosis, theileriosis, tularemia, tick born encephalitis and severe fever with thrombocytopenia syndrome (SFTS). Ticks are obligate ectoparasites of vertebrates and requires the blood for reproduction. The life cycle of I. scapularis consist of three stages namely: larva, nymph and adult. The molecular and cellular events occurring during the stages of the tick and/or of a tick parasite, without limitation, a babesia parasite or B. microti, are yet to be determined and progress is greatly impeded by existing hurdles in isolating parasites from the infected ticks.

In embodiments, a transfected cell or organism which expresses a reporter gene, e.g., without limitation, a cell line, parasite, or pathogen, can be easily visualized, detected and monitored in these cells or organisms by fluorescence microscopy, thus overcoming these hurdles. Similarly, in embodiments, reporter gene expressing parasites (e.g. GFP, mCherry, or others) can be easily sorted by fluorescence activated cell sorting (FACS). FACS is a tool that allows to isolate the reporter gene expressing cells from cells that do not express the reporter gene. FACS can also be used in cases where the parasite infection rate is very low to easily sort parasite infected cells from non-infected cells.

The identification of key biological molecules in either the tick vector, the parasite, or both, can serve as a target of intervention strategies and control of tick-borne diseases. The cellular and molecular events that occur during the tick stages of a parasite, such as, without limitation, a babesia parasite, e.g., without limitation, B. microti are yet unknown and impeded by the complex life cycle of parasite.

In embodiments, to study the life cycle and e.g. the process of gametogenesis, parasite fertilization, zygote formation, oocyst development, sporozoite formation, maturation and their migration from midgut via coelomic fluid to salivary gland, as well as the pathogenesis of a parasite (such as, without limitation, babesia, e.g. B. microti) in tick vector and mammalian host the following procedure may be used: confocal and intravital microscopy is performed on a reporter-gene expressing parasite, as will be apparent to a person of ordinary skill. For example, the parasite will be infected with a vector containing a reporter gene of choice, for example, without limitation, GFP, and optionally a gene of interest for overexpression. Parasites expressing reporter genes only may be used to determine the parasite burden and to determine the development, replication and growth of the parasite either in an in vitro or in vivo system, as will be apparent to a person of ordinary skill, or essentially as described by Miller et al. (2013), Quantitative Bioluminescent Imaging of Pre-Erythrocytic Malaria Parasite Infection Using Luciferase-Expressing Plasmodium yoelii, PLoS ONE 8(4): e60820 (e.g. as published online at https://doi.org/10.1371/journal.pone.0060820), which hereby is incorporated herein by reference in its entirety.

In embodiments, a screening procedure for a new drug for therapy or cure of a disease, e.g., without limitation, a parasite related disease, e.g. babesiosis, is provided. To screen for a drug, in a first step, a reporter-gene expressing parasite is provided, e.g., without limitation, a reporter or fluorescent gene expressing parasite, which in a second step, in case of the fluorescent gene, may be visualized by fluorescence microscopy and in a third step the parasites are counted, thus determining an effect of a drug on parasite growth and replication. Generally, the parasite numbers in the presence and in the absence of a drug will be compared, thus identifying a suitable drug and relative strength thereof, e.g. of inhibiting parasite growth.

In embodiments, the third step of screening may be performed by fluorescent activated cell sorting (FACS). The vectors described herein in combination with FACS advantageously allow to screen for multiple drugs in a short period of time.

In embodiments, alternatively, a parasite transfected with a luciferase gene can be used to determine the parasite load of a host, e.g., without limitation, a mammal, a human, a vector, or a tick, in in vitro and/or in vivo conditions using an IVIS imaging system, for example IVIS Lumina II® animal imager (PerkinElmer®, Waltham, Mass.) or IVIS Spectrum® (PerkinElmer®, Waltham, Mass.). In a first step, a bioluminescent parasite is provided, e.g. a parasite expressing luciferase. In a second step, an organism is infected with the bioluminescent parasite. In a third step, the organism is administered with one or more drugs. In a fourth step, after administration of a substrate, e.g., without limitation, D-Luciferin, an imager is used on the infected organism to determine the effect of the one or more drug on the bioluminescence signal formed by gene expression of luciferase which converts D-Luciferin into a detectable bioluminescent signal. The procedure allows 1000 or more drug compounds to be tested, screened and/or compared. Thus the parasite load in in vitro and in vivo systems may be compared; the procedure may also be performed, for example, as described by Miller et al. (2013), Quantitative Bioluminescent Imaging of Pre-Erythrocytic Malaria Parasite Infection Using Luciferase-Expressing Plasmodium yoelii, PLoS ONE 8(4): e60820; (e.g. as published at https://doi.org/10.1371/journal.pone.0060820), which hereby is incorporated herein by reference in its entirety.

In embodiments, the tick stage biology of a Babesia parasite may be determined as follows. Transfected B. microti expressing one or more reporter gene, for example, without limitation, GFP and mCherry, can be used to track parasite development in ticks by using confocal microscopy. In a first step, the ticks are infected with transfected B. microti by exposing the ticks to the parasite, as will be apparent to a person of ordinary skill. After infection, the ticks are mounted on a glass slide and confocal imaging is performed to determine the parasite development and track it over time. B. microti stages include: gamete formation, fertilization, zygote formation and others, as will be apparent to a person of ordinary skill. These stages can be followed due B. microti reporter gene expression, e.g. fluorescence of GPF and mCherry, using fluorescence microscopy of the appropriate wave length, as will be apparent to a person of ordinary skill.

In embodiments, B. microti parasites transfected with a reporter gene may advantageously be used for drug screening, including, for example, in vitro or in vivo drug screening, e.g. as described herein-below, e.g. in examples 6, 7, and 8. Reporter genes may be, e.g. fluorescent or bioluminescent reporter genes, which may be detected by appropriate methods such as imaging, including, without limitation, fluorescent or bioluminescent imaging. For example, luciferase converts D-Luciferin into a detectable bioluminescent signal. This and the other screening procedures described herein allow a large number, e.g., without limitation, 1000 or more drugs (chemical compounds, organic or inorganic substances, isolates, extracts, or mixtures thereof), to be tested, screened and/or compared. Thus e.g. the parasite load may be compared in in vitro or in vivo systems. Alternatively, a drug screening procedure may be performed, for example, as described by Miller et al. (2013), Quantitative Bioluminescent Imaging of Pre-Erythrocytic Malaria Parasite Infection Using Luciferase-Lxpressing Plasmodium yoelii, PLoS ONE 8(4): e60820 (e.g. as published at https://doi.org/10.1371/journal.pone.0060820), which hereby is incorporated herein by reference in its entirety.

The foregoing summary of the present invention with the preferred embodiments should not be construed to limit the scope of the invention. It should be understood and obvious to one skilled in the art that the embodiments of the invention thus described may be further modified without departing from the spirit and scope of the invention.

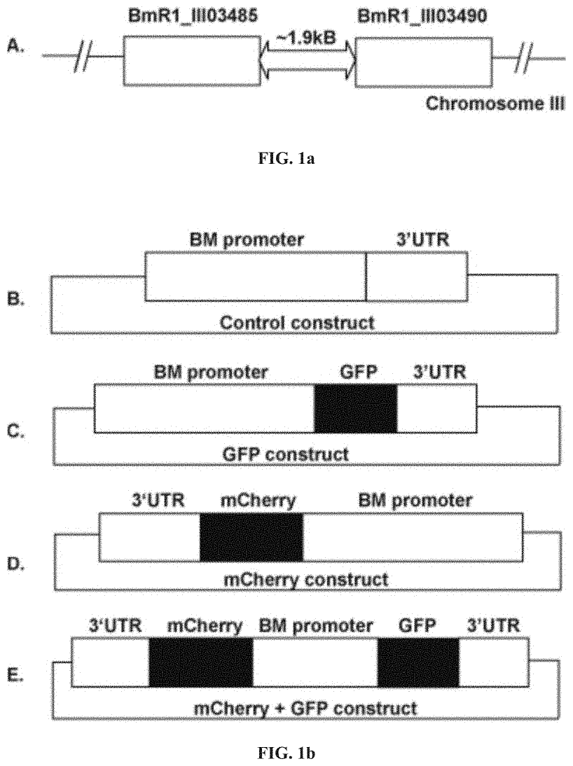

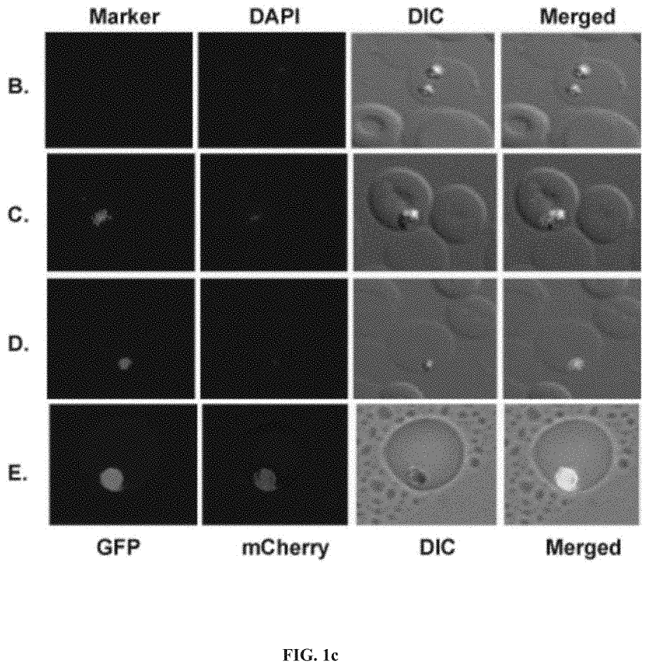

Turning to FIG. 1 a, FIG. 1b and FIG. 1c, transfection of a variety of multifunctional constructs show multifunctional expression of fluorescent reporter genes (GFP and mCherry, green and red, respectively) located downstream and upstream of the promoter. The constructs are shown schematically in panels A-E of FIG. 1b, while FIG. 1a shows the genomic locus of the BM-CTQ41297 promoter (SEQ ID NO:1). The corresponding photographs in FIG. 1c show corresponding gene transcription signals determined by fluorescence microscopy for B. microti parasites transfected with either a negative control plasmid (panel B), GFP construct (panel C), mCherry construct (panel D), and a bifunctional GFP/mCherry construct (panel E). In all panels B-E, the DIC (differential interference contrast) images show a parasitized Red Blood Cell (RBC), here parasitized by B. microti, and a merged image shows an overlap of all images shown. In panels C-E, green and red fluorescence signals correspond to the same parasite expressing GFP and mCherry markers, respectively, and blue DAPI staining corresponds to the nucleus of the parasite within the RBC.

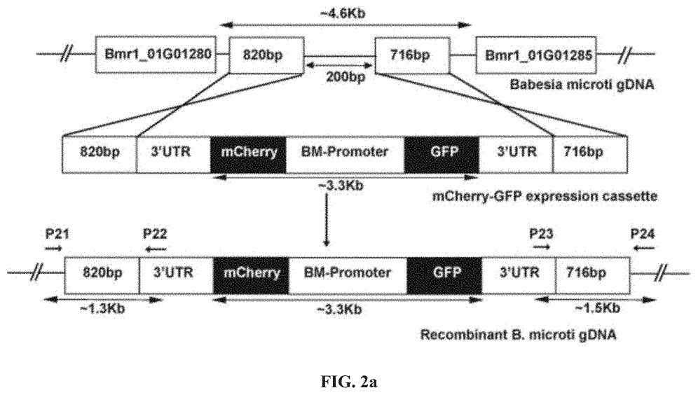

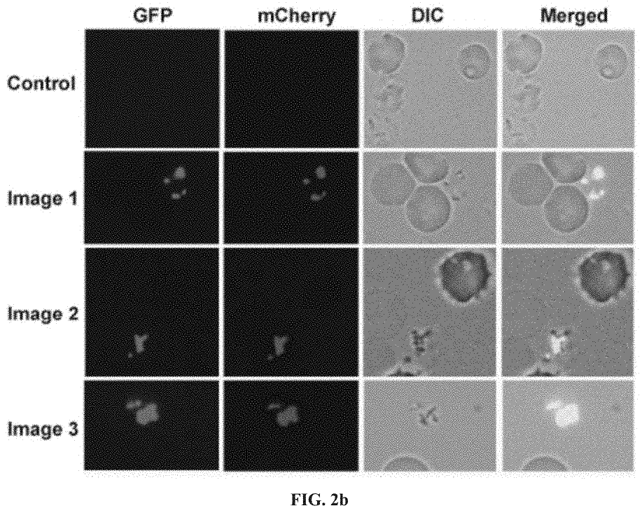

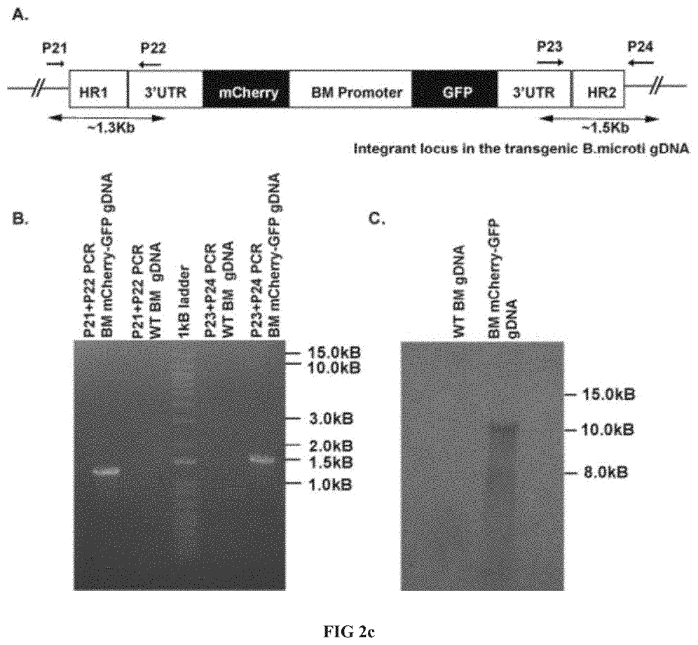

In FIG. 2a and FIG. 2b, the results of a stable transfection of a multifunctional construct into a parasite (here: B. microti) are shown. FIG. 2a shows a schematic representation of genomic parasite DNA (top) and a construct with babesia promoter before (middle) and after (bottom) stable integration into the genomic DNA (here of B. microti); the construct is bifunctional and carries two genes (here: GFP and mCherry fluorescent markers with a 3′UTR), downstream and upstream of the promoter, respectively. The construct also contains upstream (820 bp, homologous region 1 or HR1) and downstream (716 bp, homologous region 2 or HR2) sequences that are specific for an integrant or recombinant locus and allows for double homologous recombination into the B. microti gDNA. The homologous region contains a nucleotide sequence of a genomic locus, also known as the integrant locus, where the construct with the expression cassette will be inserted. In FIG. 2b, photographs show fluorescent signals of Babesia microti-infected RBCs. In the columns, GFP and mCherry correspond to green and red fluorescence signals in the parasite (here: B. microti), respectively, observed by fluorescence microscopy, DIC (differential interference contrast) images show a microscopic image of a cell preparation, and merged images show an overlap of all images shown.

In FIG. 2c, the results of a diagnostic PCR performed as described in example 6 is shown which demonstrates successful integration of the construct present in transfected B. microti formed as described in example 2. In panel A of the figure, P21 and P22 represent the primers used to show successful 5′ integration, and similarly, P23 and P24 represent the primer pair that demonstrates 3′ integration. Panel B shows the diagnostic PCR results that confirm the integration of reporter genes in the B. microti parasite at right locus by showing PCR products of expected sizes. In panel C, a Southern Blot analysis of wildtype (WT) and transgenic B. microti parasite gDNA is shown which detects the expected band of 10.5 kB from the restriction enzyme-digested GFP-containing construct that was integrated in the transgenic parasite using a GFP probe; no band is detected in the WT parasite gDNA (which does not contain the GFP construct).

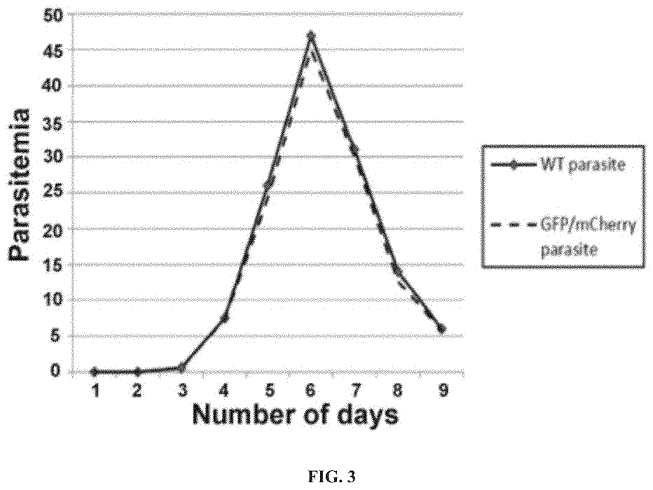

In FIG. 3, a growth curve analysis shows that growth and parasitemia of wildtype (WT) and transfected parasites are similar. Transgenic parasites (here: B. microti) with a bifunctional construct are subjected to blood stage parasitemia examination to determine the quantitative content of parasites and the growth of the parasite load in the organism over time. For example, here the blood taken from the tail of infected mice was smeared on the thin slides and fixed with methanol, then stained with Giemsa stain. The blood stage parasites are counted daily to determine the parasitemia. The results show both WT and transgenic parasite have a nearly identical growth curve with a maximum parasitemia of about 48% around day 6 post infection. The results demonstrate normal growth and successful integration of the transfected genes (here: GFP and mCherry transfected into B. microti parasite using a promoter construct as described herein).

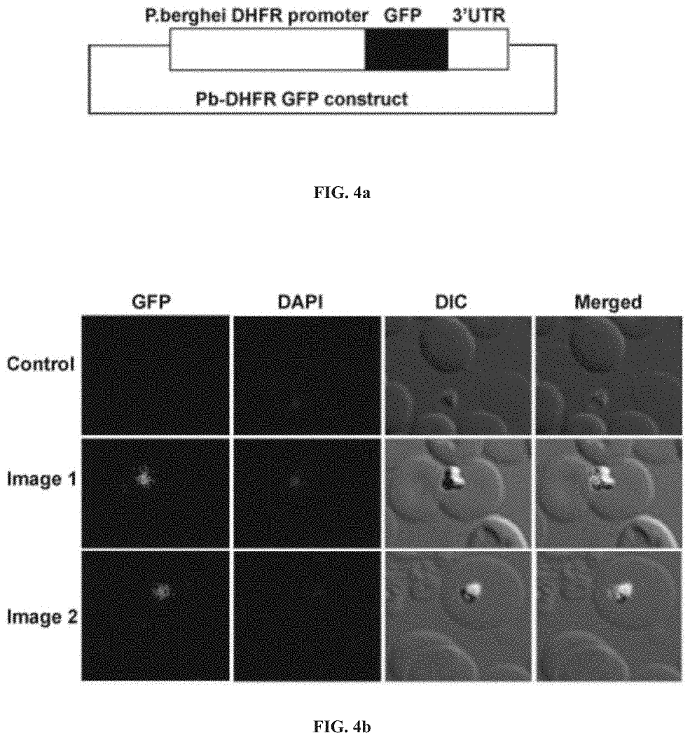

In FIG. 4a and FIG. 4b, for the first time, successful transfection and expression of a Plasmodium promoter construct (here: P. berghei) into a Babesia parasite (here: B. microti) is shown. FIG. 4a shows a schematic representation of the construct which is a standard cloning vector such as Bluescript (pBSK) with a GFP fluorescent reporter gene insert under control of the DHFR promoter from P. berghei, and a 3′UTR element. FIG. 4b shows photographs of microscopy results, each column either fluorescent (GFP/green for B. microti in RBC or DAPI/blue, corresponds to the nucleus of B. microti), DIC, or merged. DIC and merged show RBCs which are B. microti infected, DAPI shows the nucleus of the parasite infecting a RBC, and the GFP columns show the signals resulting from GFP expression under control of the Plasmodium DHFR promoter in a B. microti parasite. The control shows the B. microti parasitized RBC (without construct) shows no signal and thus no expression.

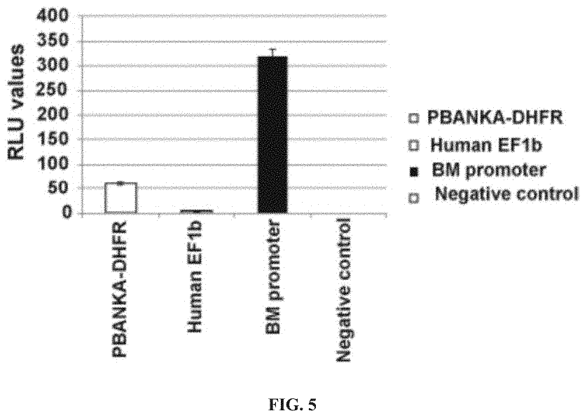

In FIG. 5, the improved strength of the Babesia promoter (here: B. microti or “BM”) compared to a strong constitutive promoter (here: PBANKA DHFR of SEQ ID NO:2) is shown. The diagram shows RLU (Relative Light Unit) values from 0 to 400 RTU, with both controls showing RTU of 0 or close to 0, PBANKA-DHFR at about 60, and the Babesia promoter at about 325 (i.e. about 5 to 6 times stronger). The results demonstrate that the Babesia promoter is capable of providing a significantly stronger expression, even when compared to a known strong constitutive promoter (here: PBANKA DHFR from a rodent malaria parasite, Plasmodium berghei).

As used herein, the term “prepatent period” is the period between infection with a parasite and the demonstration of the parasite in the body, especially as determined by the recovery of an infective form from a bodily fluid, e.g. from the blood.

As used herein, the term “expression system” comprises artificially engineered DNA in transfectable form and minimally comprises a promoter and one or more elements for propagation in a host cell or organism; for example, generally, the DNA further comprises one or more open reading frame (ORF) operatively linked to the promoter, e.g. to test for expression of the translated product of the ORF (if any). For example, a single vector may constitute or be part of a particular expression system. Typically, a vector contains one or more elements for its propagation and for the expression of the ORF comprised in the vector in one or more types of host cells. The vector may optionally be provided in its transfected form, e.g. transfected into a host cell or host organism. An expression system may be transient or stable, e.g. a transfected cell line wherein the DNA is stably propagated, e.g. by a self-replicating virus or plasmid or integration into the genomic DNA.

As used herein, the term “vector” is an engineered recombinant DNA molecule used as a vehicle to artificially carry genetic material such as an ORF, typically foreign material, into another cell, where it can be replicated and/or expressed. Four major types of vectors include plasmids, viral vectors, cosmids, and artificial chromosomes. Vectors typically include one or more propagation or expression elements, e.g., without limitation: an origin of replication, origin of plasmid replication (oriP), a multicloning site, one or more selectable marker or reporter gene (e.g. fluorescent, antibiotic resistance), one or more ORF, CDS or gene for expression of a target protein, regulatory elements to regulate protein expression, and signal elements such as, e.g., polyadenylation signal, glycosylation signal (for example for expression of glycosylated proteins in e.g. mammalian cells).

As used herein, the term Open Reading Frame or “ORF” is the part of a reading frame that has the ability to be translated. Typically an ORF is a continuous stretch of codons that begins with a start codon (usually AUG) and ends at a stop codon (usually UAA, UAG or UGA). An ATG codon (AUG in terms of RNA) within the ORF (not necessarily the first) may indicate where translation starts. The transcription termination site is located after the ORF, beyond the translation stop codon. An ORF may contain introns. In eukaryotic genes with multiple exons, introns are removed and exons are then joined together after transcription to yield the final mRNA for protein translation.

As used herein, the term Coding Sequence or “CDS” is the region of DNA that is translated to form proteins. In contrast to the ORF, which may contain introns and contains start and stop codons, the CDS refers to those nucleotides that can be divided into codons and which are translated into amino acids by the ribosomal translation machinery.

### EXEMPLARY EMBODIMENTS

### Example 1—Transient Transfection of B. microti Parasites with Different Promoter Constructs

A transfection of parasites with a parasite promoter (here: B. microti & SEQ ID NO:1) is performed as follows. A schematic representation of the genomic location of the B. microti promoter of SEQ ID NO:1 (BM-CTQ41297 promoter) is shown in FIG. 1a. As shown in the figure, the promoter is flanked by the two different genes. The episomal constructs may be based on a standard cloning vector such as Bluescript (pBSK (+)) and modified for expression accordingly; the constructs used are shown schematically in FIG. 1b. As a negative control (B), B. microti parasites transfected with a blank vector without reporter gene insert (but with BM-CTQ41297 promoter and 3′UTR, compare SEQ ID NO: 35). C shows GFP and the 3′UTR downstream of the BM-CTQ41297 promoter, D shows the 3′UTR and mCherry upstream of the BM-CTQ41297 promoter, E shows a construct with both genes and 3′UTR, mCherry upstream and GFP downstream, respectively. Electroporation for both genes is performed as follows. The construct DNA for transfection is prepared using a midi kit (Qiagen®, Hilden, Germany) according to the manufacturer's instructions. Briefly, 100 μl of B. microti infected blood (10% parasitemia) is collected from infected mice in a 1.5 ml tube with 10 μl of heparin. The infected blood is centrifuged at 1000 rpm at room temperature (RT) and washed once with RPMI medium. Approximately 20 μl of the infected RBCs pellet is mixed with 90 μl of the transfection buffer (Lonza, Basel, Switzerland, VAPA-1002, Human T Cell Nucleofector™ kit) and 10 μg of construct DNA. The electroporation is performed in a 1 cm cuvette selecting manufacturer's pre-programmed protocol U033 in a Nucleofector 2B® device, (Lonza, Basel, Switzerland, VAPA-1002) according to the manufacturer's instructions or Gene and Pulser II® (Bio-Rad®, Hercules Calif.; for example, without limitation, with electroporation settings at 1.3 kV/25 μF/200×). 100 μl of complete RPMI medium is immediately added to the transfected parasites and the transfected parasites are injected into mice, here C57BL/6J mice, via intravenous injection. In a control experiment for comparison, parasites are transfected with 10 μg of control plasmid DNA wherein the control plasmid does not contain GFP or any other marker gene, and otherwise treated as described above.

After electroporation, the parasites are immediately injected into C57BL/6J mice and after two days of transfection analyzed by fluorescence microscopy as follows: a drop of blood is collected in a 1.5 ml tube containing heparin from the tail of mice infected with transfected parasites. The blood is diluted in phosphate buffer saline (PBS) in a ratio of 1:100. 20 μl of the diluted blood is mounted on a glass slide with a glass coverslip. The transfected parasites are analyzed by fluorescence microscopy to check if transfected parasites express the reporter gene (here: GFP) using a fluorescence microscope, e.g. Axio Imager M2®, Zeiss®, Dublin, Calif.). The transfected parasites are reporter gene (e.g. here: GFP) positive as confirmed by fluorescence microscopy and shown in FIG. 1c, see C. In a control experiment, the parasites are transfected with the episomal construct without a gene which serves as a negative control; these parasites do not show a fluorescent signal when analyzed by fluorescence microscopic analysis, as shown in FIG. 1c, B. The results for gene transfection constructs show that Babesia-based promoters such as the BM-CTQ41297 promoter can be used to express one or multiple reporter gene in parasites, including in particular B. microti parasites. Transfection of an episomal construct with the GFP gene at a downstream location of a Babesia-based promoter, e.g., without limitation, the BM-CTQ41297 promoter for transfection of a B. microti parasite (FIG. 1b, c, C) shows that the promoter can strongly express a reporter gene inserted at the downstream region of the promoter. Similarly, transfection of an episomal construct with the mCherry gene at an upstream location of a Babesia-based promoter such as the BM-CTQ41297 promoter (FIG. 1b, c, D) shows that the promoter can strongly express a reporter gene inserted at the upstream region of the promoter. Transfection of an episomal construct with both the mCherry gene at an upstream location of the promoter and with the GFP gene at a downstream location of the promoter (FIG. 1b, c, E) shows that the promoter can strongly express a reporter gene inserted at both upstream and downstream regions of the promoter simultaneously. These results show that a Babesia-based promoter, for example, without limitation, the BM-CTQ41297 promoter can strongly express a gene located either downstream or upstream of the promoter, or both. This bifunctional promoter activity is useful to express two markers simultaneously in the parasites and has been shown in B. microti parasite for the first time. For example, the bifunctional and/or bidirectional promoter (which may express a gene placed on either end of the promoter, e.g. two genes, one or more upstream of the promoter, and one or more downstream of the promoter, e.g. two genes of one or more function) may advantageously be used for simultaneous expression of two or more genes of interest, e.g. to analyze the co-localization of two proteins within an organism or within cells at the same time, and/or to express a drug resistance marker and a fluorescence marker simultaneously.

### Example 1b—Electroporation of B. microti

The DNA construct is prepared as described in example 1, and linearized with a suitable enzyme, as will be apparent to a person of ordinary skill, resulting in linearized construct DNA. Approximately 10 μg of the linearized construct DNA is introduced into B. microti parasites using the U033 program of the Nucleofector2B® device (Lonza, Basel, Switzerland). A BALB/C or C57BL6/J mouse is intravenously infected with B. microti (here: B. microti grey strain, ATCC™ 30221D). Parasitemia is analyzed daily by thin blood smear, taking a small blood sample from the tail of infected mice. 100 μl of the blood is collected from B. microti infected mice (10% parasitemia) in a 1.5 ml Eppendorf tube containing heparin and washed twice with incomplete RPMI medium and centrifuging at 1000 rpm for 2 minutes. The human T-cells Nucleofector® kit (VAPA-1002, Lonza, Basel, Switzerland) is used for the B. microti parasite transfection or eletroporation. First, 18 μl of solution ‘A’ and 82 μl of solution ‘B’ are mixed in a tube and kept on the ice. 10 μg of the linearized construct digested with SAP1 restriction enzyme is added to the solution. Next, 20 μl of packed red blood cells (RBC) with 10% parasitemia is added to the transfection mixture per transfection. The electroporation is performed using the U033 program in a Nucleofector-2B® machine (Lonza, Basel, Switzerland) or a Gene and Pulser II® (Bio-Rad®, Hercules, Calif., with electroporation settings at 1.3 kV/25 μF/200×), and 100 μl of incomplete RPMI is added to the electroporated parasites which are immediately injected into the mice. As control, a plasmid construct without “homologous arm” (compare example 2 and FIG. 2a) and without a reporter gene is used. The construct-injected mice are monitored daily for parasitemia using thin blood smear (Giemsa method, e.g. as described in example 3 herein-below). After 48 hours, the transfected parasite is analyzed by fluorescence microscopy to visualize reporter-gene expressing parasites (here: GFP).

### Example 2—Stable Integration of Promoter Constructs into B. microti Parasites

To perform a stable integration of GFP and mCherry reporter genes in a Babesia parasite (here: B. microti), a targeting construct (plasmid DNA containing a sequence homologous an upstream region of the integrant locus, here of about 823 bp, and a sequence homologous to a downstream region of the integrant locus, here of about 716 bp for double homologous recombination, the promoter, and the reporter genes), containing a bifunctional Babesia-based promoter such as the BM-CTQ41297 promoter along with GFP and mCherry reporter genes is prepared; see FIG. 2b for schematic representation of the construct. The targeting construct is a pBluescript SK+ vector (pBSK+) linearized with a suitable restriction enzyme (here: Sap1 restriction enzyme), and introduced into the parasites, e.g. by electroporation.

The transfection/electroporation may be performed as follows: transfect 10 μg of linearized construct DNA into a parasite (here: B. microti) using the manufacturer's U033 program in a Nucleofector2B™ (Lonza, Basel, Switzerland). 100 μl of blood is collected from parasite (here: B. microti-infected mice, 10% parasitemia) into a 1.5 ml Eppendorf tube containing heparin; the mixture is washed twice with incomplete RPMI media by after centrifuging at 1000 rpm for 2 minutes each time. Human T-cells Nucleofector™ kit (VAPA-1002, Lonza, Basel, Switzerland) is used for the parasite (here: B. microti) transfection. First, 18 μl of solution ‘A’ of the kit and 82 μl of solution ‘B’ of the kit are mixed in a tube and kept on the ice. 10 μg of the linearized construct is added to the solution. Next, 20 μl of packed RBC with 10% parasitemia are added to the transfection mixture per transfection. The transfection is performed using the manufacturer's U033 program in a Nucleofector-2B™ machine (Lonza, Basel, Switzerland) or Gene and Pulser II (Bio-Rad, e.g., without limitation, with electroporation settings at 1.3 kV/25 μF/200×), and 100 μl of incomplete RPMI is added to the transfected parasites. The resulting mixture is immediately injected into the mice. In a control experiment a plasmid without homologous arm and without a reporter gene is used as a control. After 48 hours, the transfected parasite is analyzed by fluorescence microscopy to visualize GFP expressing parasites. The GFP expressing parasites are sorted by FACS and immediately injected into a mouse to enrich the transfected parasites.

The construct may be formed and inserted into a preferred genomic locus/region as follows: a GFP expression cassette is inserted to replace a 200 bp region present in an intergenic sequence between Bmr1-11102355 and Bmr-11102360 parasite genes. The construct for stable transfection contains a ˜820-bp fragment from the upstream region of 200 bp of intergenic sequence and is amplified by PCR using primer P17 (SEQ ID NO: 24) and P18 (SEQ ID NO: 25). This fragment is cloned between the Kpn1 and Xho1 sites of the GFP/mCherry construct which is used in the transient transfection as described herein. A ˜716-bp fragment from the downstream region of a 200 bp in intergenic sequence is amplified by the PCR using primers P19 (SEQ ID NO: 26) and P20 (SEQ ID NO: 27). The nucleotide sequence of the primers is provided in the sequence protocol and in the table below. The fragment is cloned between Not1 and Sac restriction sites in a GFP/mCherry construct plasmid as described herein. The region is selected because the size of an intergenic sequence between these two genes is ˜4.6 Kb and the deletion of 200 bp nucleotides in the middle of this intergenic sequence does not interfere with the expression of adjacent genes in the parasite. Analysis of the 200 bp DNA including bioinformatics analysis shows it does not contain a conserved motif or a promoter sequence. The GFP expression cassette for stable transfection is described in detail in the transient transfection described herein-above. 10 μg of construct DNA is linearized by restriction digestion using Sap1 enzyme. The linearized construct DNA is precipitated and suspended in 10 μl of sterile water, and can then be utilized for transfection, e.g. by electroporation as described herein-above.

The electroporated parasites may then be injected into a suitable host animal, e.g. a mammal, for example they are immediately injected into mice. Injection/infection is followed by analysis of the relevant gene or genes, here the two fluorescent reporter genes; such analysis may be cell based, e.g. by fluorescent analysis of a cell preparation. For example, two days after transfection the red blood cells (RBC) of the mice are analyzed by fluorescence microscopy to observe reporter gene expression in parasitized RBC, see FIG. 2b. Fluorescent activated cell sorting (FACS) is used to sort cells, i.e. here RBC, having GFP and mCherry positive parasites. Results show expression of both GFP and mCherry proteins in the same parasite, demonstrating stable transfection of the genes in a Babesia parasite, e.g. a B. microti parasite. Southern blot analysis and diagnostic PCR confirms successful integration of the GFP and mCherry genes into the parasite, as shown in FIG. 2c.

Alternatively/additionally, a double homologous recombination method may be performed for example as described by Jailyan et al., J. Biol. Chem. 2015 Aug. 7; 290(32):19496-511, which hereby is incorporated herein by reference in its entirety.

The results of example 2 demonstrate stable transfection of the B. microti parasite is achieved and thus for the first time a locus for the genetic manipulation of the parasite is identified; this locus is located in the intergenic region of Bmr1-11102355 and Bmr-11102360 genes. The nucleotide sequence from the 468280-468480 position in the B. microti genome is replaced with a targeting construct containing a promoter and the GFP and mCherry expression cassette as described herein-above, also shown in FIG. 2a and FIG. 2b; also compare description thereof. The identified locus can be used to integrate any foreign gene into a B. microti parasite without affecting the growth of the parasite. Example 2 shows successful insertion of a foreign gene at this locus that did not affect the parasite life cycle; the transgenic parasites are similar in growth as compared to the parental/wildtype (WT) parasite.

### Example 3—Growth Analysis and Parasitemia of Wildtype and Stably Transfected Parasites

A growth curve analysis of the transgenic parasites (here: B. microti) expressing reporter genes is performed by blood stage parasitemia examination. Parasitemia is the quantitative measurement of parasites in the blood. It is used as a measurement of parasite load in the organism and an indication of the degree of an active parasitic infection. The mice are injected with 10,000 of transgenic and WT parasites in a group of 10 mice each. Parasitemia is determined for both the wildtype (WT) parasite and the parasite stably transfected with a bifunctional construct (here: GFP/mCherry); the blood stage parasitemia is determined as follows: the blood stage parasitemia is determined by the giemsa method as follows: a thin smear of blood collected from the tail vein of infected mice is made on the glass slide, fixed with methanol and stained with giemsa stain for about 15 min. The parasite numbers in the blood of infected mice are determined by bright field, 100× objective microscope. Parasitemia is measured daily. The prepatent period, i.e. the first appearance of the parasite in the blood of infected mice, is determined in case of WT and transgenic parasites. The growth curve of WT and transgenic B. microli parasite is determined by the daily measuring of parasitemia over a period of about 8-9 days. Results are shown in FIG. 3 (a scale of 0 to 60% parasitemia correspond to the total number of parasite-infected RBC in percent. As shown in FIG. 3, the growth and parasitemia are similar, with both WT and transgenic parasite having a maximum parasitemia of about 48%. Similarly, the prepatent period of WT and transgenic parasite (determined by the parasitemia of infected animals) is similar, as shown in the table below.

| | Percentage of | Prepatent period |
|---|---|---|
| Parasite | Parasitemia on day | (Number of days) |
| WT | 0.005 | 0 |
| GFP | 0.005 | 0 |

As shown in the table, the prepatent periods showed no difference. This demonstrates that the transgenic parasites expressing GFP and mCherry do not have any defect and behave like WT parasites in their growth and other features.

The results of example 3 demonstrate normal growth and successful integration of the transfected genes (here: GFP and mCherry) in the parasite (here: B. microti). Further, these results demonstrate selection of the right genomic locus for a stable transfection of the B. microti parasite and parasites are growing normally even after successful integration of fluorescent markers. This locus may preferably be as follows: insertion of the GFP and mCherry expression cassette as described herein to replace a 200 bp region present in an intergenic sequence between Bmr1-11102355 and Bmr-11102360 parasite genes. The nucleotide sequence from 468280-468503 position in the B. microti genome are replaced with the targeting construct containing GFP and mCherry expression cassette, also compare example 2, and see FIG. 2b. Other loci may be possible, and may be tested for successful integration and normal infection and growth as described herein, as will be apparent to a person of ordinary skill.

### Example 4—Promoter (Here: DHFR) from Plasmodium is Expressed in Babesia

The DHFR promoter is a strong promoter which originates from another apicomplexan parasite (Plasmodium berghei or “Pb”, including PbANKA) which is shown to be recognized in Babesia parasites (here: B. microti), see FIG. 4a and FIG. 4b. For the DHFR construct, a ˜2.2 Kb region of 5′UTR of the PbDHFR gene is cloned into a suitable vector e.g. pBSK(+) or Simple-Amp between its Xho1 and EcoR1 restriction site. For cloning, first, the 5′UTR of PbDHFR gene is amplified from PbANKA gDNA using primers P13-Xho1 and P14-EcoR1 and cloned into the vector, for the pBSK vector e.g. at the Xho1 and EcoR1 sites. Next, the GFP gene is cloned between the vector's EcoR1 and BamH1 sites in a pBSK vector containing the DHFR promoter from PBANKA. The GFP gene is amplified using primers P15-EcoR1 and P16 BamH1. At 3′end of the gfp gene, ˜627 nucleotides from the 3′UTR of the B. microti gene are cloned for proper termination of the gfp-mRNA. The 3′UTR of B. microti is amplified by PCR from B. microti gDNA using P5-BamH1 and P6-Not11 primers and cloned into the plasmid, e.g. the pBSK(+) plasmid at its BamH1 and Not1 restriction sites. Transfection and infection of mice is performed essentially as described for example 1 above (transient transfection). Results are determined by fluorescence microscopy of B. microti infected RBC as described in example 1 above. The results determine expression of GFP in an apicomplexan Babesia parasite under the control of a constitutive promoter from an apicomplexan Plasmodium parasite, as shown in FIG. 4b.

### Example 5—Babesia Promoter Outperforms PbANKA-DHFR

The strength of a Babesia-based promoter (for example, without limitation, the BM-CTQ41297 promoter of SEQ ID NO:1) and that of a strong constitutive promoter (here: PBANKA DHFR, SEQ ID NO:2) are determined and compared, representative results are shown in FIG. 5. A promoter (SEQ ID NO:1, from B. microti or “BM”) is compared to PBANKA-DHFR (known to be a strong constitutive promoter), a blank/negative control, and, as a further negative control, human EF1b promoter. The promoter constructs are prepared as described in examples 1 and 4, respectively. Relative Light Units (RLU) are measured by luminometer. The results showed that the BM-CTQ41297 promoter can outperform strong promoters such as PBANKA-DHFR. Similarly the constitutive promoter human EF1b is outperformed by a Babesia-based promoter, including e.g. by the B. microti promoter (as shown in FIG. 5). As determined by reporter gene analysis, the Babesia promoter is at least about 5 to 6 times stronger compared to PBANKA-DHFR (a promoter derived from a rodent malaria parasite Plasmodium berghei with strong constitutive activity commonly used for the expression of reporter genes in various apicomplexan parasites), see results shown in FIG. 5.

### Example 6—In Vitro Drug Screening Assay

The reporter-gene transfected B. microti parasite cell lines may be used in drug screening. For example, the host cells may be cultured in a suitable receptacle for cell culture and assaying, e.g. in a well plate of suitable size, e.g. 24 or 48 well plate in a suitable incubator, e.g. CO₂ incubator under appropriate conditions (humidity, temperature, etc.). After sufficient cell numbers are reached, the host cells will be infected with the transfected B. microti parasite cell line expressing one or more reporter gene (e.g. GFP or luciferase, etc.). The one or more drug to be screened (e.g. chloroquine, primaquine or any newly synthesized or previously existing drug) can be added at one or more different concentration to the infected host cells. After addition of the drug, the drug-exposed infected host cells and, for comparison, infected host cells not exposed to the drug, are monitored or tracked, e.g. by measuring their fluorescence due to the reporter gene (e.g. GFP, luciferase, etc.). For example, monitoring may be performed qualitatively or quantitatively, e.g. by determining the number of GFP positive cells using fluorescence microscopy. Alternatively/additionally, the parasite load of the infected host cells may also be determined, and optionally quantified, by IVIS imaging, e.g. using luciferase substrate. In a control and for comparison, the parasite and/or parasite-infected host cell will not be exposed to the drug. For IVIS imaging, suitable equipment includes, e.g., without limitation, IVIS Lumina II® animal imager or IVIS Spectrum® (both PerkinElmer®).

### Example 7—In Vivo Drug Screening Assay

The in vivo screen may be performed essentially as described in example 6, except that the cells are present in form of an organism or subject, e.g. an animal, including, e.g. a human. One or more subject will be infected with the transfected Babesia microti parasite expressing a reporter gene, in particular, e.g. the luciferase gene. The one or more infected subjects will be treated with a drug, including e.g. a chemically synthesized drug, a nature-derived substance or mixture thereof. A subject not treated with or exposed to the one or more drug is used as an infection control. Then a relevant parameter such as e.g. the parasite load will be determined in the subject by measuring the activity of the reporter gene, e.g. fluorescence, for example, by performing a luciferase imaging using IVIS, as described in example 6 herein-above. The parameter, e.g. parasite load, of drug-exposed infected subjects is compared to that of the infected subjects not exposed to the drug.

### Example 8—In Vivo Drug Screening Assay Based on Bioluminescence

Alternatively, the following procedure may be performed: In a first step, a bioluminescent parasite, for example, without limitation, a Babesia microti parasite is provided by transfection with an appropriate reporter gene, for example a parasite expressing a reporter gene such as luciferase which provides bioluminescence (compare examples herein-above). In a second step, an organism is infected with the bioluminescent B. microti parasite. In a third step, the organism is administered with one or more drug. In a fourth step, after administration of a suitable substrate, e.g., without limitation, D-Luciferin, an imager is used to determine the effect of the one or more drug on the bioluminescence signal in the infected organism formed by gene expression of luciferase.

### Example 9—Electroporation of B. microti

B. microti infected blood may be used fresh or alternatively in stored form, e.g. thawed after storage in liquid nitrogen; the blood may be injected into an animal, e.g. a mouse, e.g. via the intraperitoneal route. Parasitemia is regularly checked, e.g. daily, e.g. by thin blood smear on a glass slide and stained with Giemsa stain. 100 μl of blood is collected from parasite (here: B. microti)-infected mice (10% parasitemia) into a 1.5 ml Eppendorf tube containing heparin; the mixture is washed twice with incomplete RPMI media by after centrifuging at 1000 rpm for 2 minutes each time. Approximately, 10-20 ug of vector DNA, prepared e.g. as described in example 1, 1b, 2 or 4, is added to a transfection buffer. As transfection buffer, e.g. BM transfection buffer (110-130 mMKCl, 0.12-0.13 mMCaCl2, 10-20 mM K2HPO4/KH2PO4, pH 7.6, 15-30 mM Hepes, pH 7.6, 1.0-2.5 mMEGTA, 2.0-6 mM MgCl2, final pH 7.6) may be used. Electroporation may be performed in any suitable electroporation equipment, e.g. in a Gene Pulser II apparatus (Bio-Rad®, ) according to the manufacturer's manual, e.g. using 0.2 cm electroporation cuvettes containing 62.5 ul filter sterilized buffer plus the circular plasmid DNA (in case of transient transfection), or alternatively, linearized DNA (in case of stable transfection), and 20 ul of washed B. microti-infected RBC (red blood cells), to a final volume of 100 ul. Electroporation is performed at a voltage of about 0.5 to about 2 kV, preferably of about 1.0 to about 1.9 kV, e.g. of about 1.3 to about 1.5 kV, and a capacitance of at about 25 uF with a time constant of about 0.1 to about 5 seconds. Without wishing to be bound by theory, it is believed that the voltage may be particularly important for successful electroporation of B. microti, which is a Babesia species that is especially difficult to transfect when compared to e.g. other Babesia species.

Alternatively, human T-cells nucleofector Kit®, VAPA-1002 (Lonza, Basel, Switzerland) may be used for transfection and electroporation of B. microti. First, 18 μl of solution ‘A’ of the kit (Lonza, Basel, Switzerland) and 82 μl of solution ‘B’ of the kit (Lonza, Basel, Switzerland) are mixed in a tube and kept on ice. 10 μg of a linearized construct, prepared e.g. as described herein-above, e.g. in examples 1, 1b or 2, is added to the solution. Next, 20 μl of packed RBC with 10% parasitemia are added to the transfection mixture per transfection. The transfection is performed using the U033 program in a Nucleofector-2B® machine (Lonza, Basel, Switzerland) and 100 μl of incomplete RPMI is added to the transfected parasites.

### Example 10—Diagnostic PCR and Southern Blot Analysis of Transfected Parasites

Stably transfected parasites are generated as described herein-above in example 2. Dilution cloning may be performed to obtain a monoclonal cell population starting from a polyclonal mass of cells. Genomic DNA of the transfected parasites obtained after dilution cloning is isolated, e.g. by blood minikit® for DNA isolation (Qiagen, Hilden, Germany). Integration specific diagnostic PCR may be performed for 5′ integration events using primers P21 and P22. Similarly, 3′ integration specific diagnostic PCR may be performed using p23 and P24 primers. These pairs of primers will give PCR product only on the integrant gDNA and not in wildtype (WT) DNA. The size of the expected PCR product is 1.3 kB for the 5′ integration site and 1.5 kB for the 3′ integration site. To further confirm integration, a Southern blot is performed to determine the integration of the reporter gene at the right locus in the parasite, e.g. the B. microli parasite. Briefly, the gDNA of the transgenic and WT parasite is digested with Eag1 restriction enzyme overnight and the resulting DNA fragments may be separated using an appropriate separation means such as an agarose gel of appropriate percentage, voltage and time, e.g. a 0.8% agarose gel at about 50 volt. The DNA is then transferred to a positively charged nylon membrane (Southern Blot) and cross linked with UV. The membrane is incubated for about 3-4 hours in a suitable prehybridization buffer at a suitable temperature, e.g. about 50 degree Celsius in a hybridization chamber. The membrane with DNA is further processed for hybridization with a suitable probe, here e.g. a DIG labeled GFP probe, for a suitable time and temperature, e.g. at about 50 degree Celsius for about 12 hours. The membrane is washed, e.g. three times with an appropriate washing buffer that removes unspecifically bound background but leaves the DNA-bound probe. The membrane is blocked with blocking buffer for a sufficient time at an appropriate temperature, e.g. about 1 hour at room temperature. A suitable anti-probe antibody is used for detection of the probe, e.g. an Alkaline phosphatase (AP) conjugated anti DIG antibody at an appropriate dilution, e.g. of about 1:5000 may be used to detect the probe. The blot is developed using an appropriate substrate for the detection system, here an enzyme-coupled antibody, e.g. a CSPD substrate which is a chemiluminescence substrate for the alkaline phosphatase enzyme. Alternatively/additionally, the Southern Blot may be performed for example as described by Jaijyain et al., J. Biol. Chem. 2015 Aug. 7, 290(32):19496-511, which hereby is incorporated herein by reference in its entirety. A band of 10.5 Kb is detected in transgenic parasite by the GFP probe, as shown in FIG. 2C. The probe is prepared using an appropriate means to label the probe, e.g. a DIG-labeling kit, according to the manufacturer's protocol and is amplified by PCR using primers P3 and P4. The results are shown in FIG. 2C

While various embodiments and examples make references to a particular component for illustrative purposes, e.g. a particular promoter sequence such as one from B. microti, and/or a particular parasite, such as a Babesia parasite or a B. microti parasite, etc., it will be apparent to a person of ordinary skill that such component may be replaced with an equivalent one achieving the same or similar effect, e.g. a homologous promoter (e.g. 60% identical, or of a percentage of identity and/or length/partial sequence as described herein) of either B. microti or of another parasite related or non-related to B. microti or Babesia which achieves a similar strength in one or more parasite. This may be easily tested with methods as described herein by comparing the relevant components. Similarly the promoter, or a promotor homologous thereto, may be used in any organism or cell thereof, including parasites and non-parasites, Babesia parasites and non-Babesia parasites, including various mammals, non-mammals, yeasts, bacteria, insect cells, mammalian cells and any other organisms cells and expression systems as described herein.

While multiple embodiments are disclosed, still other embodiments of the present invention will become apparent to those skilled in the art from this detailed description. There may be aspects of this invention that may be practiced without the implementation of some features as they are described. It should be understood that some details have not been described in detail in order to not unnecessarily obscure the focus of the invention. The invention is capable of myriad modifications in various obvious aspects, all without departing from the spirit and scope of the present invention. Accordingly, the drawings and descriptions are to be regarded as illustrative rather than restrictive in nature.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

1. A heterologous expression system comprising a promoter sequence having 60% or more identity to SEQ ID NO:1 or a contiguous part thereof, wherein the contiguous part thereof has a length of 100 nucleotides or more.

2. The expression system of claim 1, wherein the promoter sequence has 60% or more identity to a sequence selected from the group consisting of: SEQ ID NO:1.

3. The expression system of claim 1, further comprising one or more ORF, CDS or gene operably linked to the promoter sequence, wherein one of the one or more ORF, CDS or gene is encoding for one or more of: a gene product, DNA coding sequence, a reporter gene product, a selection marker gene product, an antibiotic resistance gene product or a combination thereof.

4. The expression system of claim 3, wherein the ORF, CDS or gene is selected from one or more of the group consisting of: gfp (encoding for Green Fluorescent protein), rfp (encoding for Red fluorescent protein), luc (encoding for Luciferase) mCherry (encoding for mCherry protein), lacZ (beta-galactosidase), Blasticidin, Dehydrofolate reductase, and cat (encoding for Chloramphenicol acetyltransferase).

5. The expression system of claim 1 wherein the promoter is bifunctional and is operably linked to two or more ORF, CDS or genes.

6. The expression system of claim 1, further comprising a host cell selected from the group of apicomplexan, bacterial, fungal, yeast, algae, vertebrate, invertebrate, mammalian, bird, insect, and viral host cell.

7. The expression system of claim 6, wherein the host cell is apicomplexan.

8. The expression system of claim 7, wherein the system expresses one or more of; GFP, mCherry, Luciferase.

9. The expression system of claim 8, wherein the vector expresses GFP and mCherry, or GFP and luciferase.

10. A method of expressing a gene product or protein in an expression system, wherein the expression system comprises a promoter operably linked to control expression of an ORF, CDS or gene, wherein the sequence of the promoter has 60% or more identity to SEQ ID NO:1, or a contiguous part thereof, wherein the contiguous part thereof has a length of 100 nucleotides or more.

11. The method of claim 10, wherein the expression system comprises a cell free system or a homologous or heterologous cell-based system, and wherein the cell-based system is selected from the group consisting of: parasite, apicomplexan parasite, B. microti parasite, bacterial, fungal, yeast, algae, vertebrate, invertebrate, mammalian, avian, insect, viral, E. coli, S. saccharomyces, virus-infected cells, Baculovirus-infected cells, insect cells infected with a virus including baculovirus, Tetrahymena thermophila, Dictyostelium discoideum, mammalian cell line, P. pasto, Salmonella.

12. The method of claim 11, wherein the expression system is a heterologous cell-based system.

13. The method of claim 11, wherein the expression system further comprises one or more reporter gene selected from the group comprising: GFP, mCherry, luciferase; wherein the expression system is comprised within a parasite; and wherein the parasite is detected in a host, or a cell thereof, by a signal derived from expression of the reporter gene;
wherein a host, or a cell thereof, is exposed to the parasite for sufficient duration and under conditions that allow for infection of one or more host cells by the parasite; and
wherein the presence or location of the parasite in the host, or a cell thereof, is determined qualitatively or quantitatively by a signal of the reporter gene detectable by a suitable detection system, the detection system comprising: confocal microscopy, fluorescent microscopy, and an in vivo imaging system for bioluminescence monitoring.

14. The method of claim 11, wherein signal detection is performed over sufficient time to determine parasite growth or developmental stages of the parasite, and wherein the stages comprise one or more of: gamete formation, fertilization, zygote formation, and the stage of parasite-infected red blood cells, the stage of parasite-infected red blood cells comprising one or more of ring stage, trophozoite stage, and gematocyte stage.

15. The method of claim 14, wherein a parasite infected host, or a cell thereof, is further exposed to one or more drug or substance; and wherein an increased or reduced signal for the drug or substance-exposed parasite infected host, or cell thereof, in comparison to a control not exposed to the one or more drug or substance is determined.

16. A method of stably transfecting B. microti, wherein the transfection is performed by electroporation.

17. A Babesia microti parasite cell line wherein the cell line comprises a heterologous expression system, and wherein the heterologous expression system comprises one or more reporter gene, and wherein the reporter gene, or a combination of two reporter genes, is selected from the group comprising: GFP, luciferase (Luc), mCherry, GFP combined with Luc, GFP combined with mCherry, and mCherry combined with Luc.

18. A method of expressing a gene product or protein in an expression system, wherein the cell line is a Babesia microti parasite cell line stably transfected with a heterologous expression system by electroporation, and the stably transfected B. microti parasite cell line expresses one or more reporter gene.

19. The method of claim 18, wherein the one or more reporter gene is selected from the group comprising: GFP, mCherry, and luciferase;
wherein the parasite is detected in a host, or a cell thereof, by a signal derived from expression of the reporter gene;
wherein a host, or a cell thereof, is exposed to the parasite for sufficient duration and under conditions that allow for infection of one or more host cells by the parasite; and
wherein the presence or location of the parasite in the host, or a cell thereof, is determined qualitatively or quantitatively by a signal of the reporter gene detectable by a suitable detection system, the detection system comprising an imaging system, and the imaging system selected from the group comprising: confocal microscopy, fluorescent microscopy, and an in vivo imaging system for bioluminescence monitoring.

20. The method of claim 18, wherein detection is performed over sufficient time to determine parasite growth or developmental stages of the parasite, and wherein the stages comprise one or more of; gamete formation, fertilization, zygote formation, sporozoite development, and the stage of parasite-infected red blood cells, the stage of parasite-infected red blood cells comprising one or more of ring stage, trophozoite stage, and gematocyte stage.

21. The method of claim 18, wherein a parasite infected host, or a cell thereof, is further exposed to one or more drug or substance; and wherein an increased or reduced signal for the drug or substance-exposed parasite infected host, or cell thereof, in comparison to a control not exposed to the one or more drug or substance is determined.


SEQUENCE LISTING (35 sequences)

SEQ ID NO: 1 - DNA, 1969 bp, Babesia microti
tgccattgttattcacgctagtgccattgttattcacgctaggtattaatgcctgtgtacagtgcctgtgcatattttggaagtagaaagccatacaccagtgcatttgagtcacctagctcataattacagagtaggatgactcacttaccaaagcaaatccaattggtttcgctaacttatatattaccactcatttaatcaactgtgaatcctacatattggctatggtaaatgaagtgaccctttcaaagttattacataattctacggtggcatttgctgtctatatttttaaagaccttcagtctgatcatagttaattctaaatgttcacatccattatcgcaaacaccgtcatttattttgtccttattttccgcatacttcgaagattatagttctatgttcgtacatttgctactcaatacgttactaacgttatataatatctgcgggaccatatctactaccctattgggggcatcctacagaagctaatttttggtacagttccttacaaccttaagttgatactcacaataacttaatcgcctattactccaacttatataggattgtactgttttaatgggggccgccaattttcgtagctcctccgtctgaatgctctcagaatgttcatccaacgttcccatatgcgtgagtttacattaagccttccctaggcagtttgttgttggaaacactttcaatattagtggcacgtatccatgcgcaaatctcacggtcctttgaccgatctatggttaagttaattgtaaattaaaactcttttcttttgtggcgaagcgaaatgtaatatatttcagagaaaacttgatatgtcacttctggtgtaactactacagttagattgcatttccactgattttgttccatttgctgcctaagattcgcaccgggttactggctaccctcgttaaaatatagctactctaggccatgaaggggcaatttggcctatttattatcttaagatactttggattccaatgtttccagccgattttttgcacatattataatttcagtttcaatttttagcgaaatcatggtggaaattaatgtagctggcaatatgcgctagaaatgtaagataagcatataactagcaagaatgctaagtatttcaaatataaaattcccatattagtcagctgaaatgctttaataattaaaaccacgcatatcgcatattatttgaacacaatttatgaatacggctagattgtcaaataagttttagatattagtttttctctacaacacgtatacaaataaattttctgtttagataaatgatgggaaacagtttgagttttgactaagagtatatgtggcattgaacattaagaaattgatggagatgttctccattatataagtcaacgaaatgataaaataaacaccattagtttcggacattaatatacgggtagatgcagtgattgaatgaaatatatgtatttaacgagagaattgggaggaaaacgaaattaaataattgagtaggtgcccgtaccagtagagacacgtaatacgctggttaaaataacaaaattgagattggaagcagaataatttagttaaacacacactcgaatatgttattggtgaacaattgttaaattaatggctagatagcaacatgtataactaataatgacaggaaattgagtaaaaactcccagaaatttaaaagtcacaagataatttataagatggccgtatccggatatattttaaaggcaatatgggagaggtggagctagggaaagatggtttgagttatgaagtgacttggacacaatttgacccaccccatataaccccattgatagttgtgacgataatcaccgtaaccgaaggaatctgaacaaatagcaaaacaaaattcagcagcctcttgaatgttagaaaaattttctaacattcaagaggctgc

SEQ ID NO: 2 - DNA, 2262 bp, Plasmodium berghei
gtcaacaataaataataaataaatattgtggaaataaaataacatataattatttttaatacattgatttcccttttatttttttaaatttcattgatataaaaatatataataataacatatatgatttcaaattaatcttttcaaaaatggtgtttatttttgtatgtttgtgtatgaattaatcacataacacatctattaaattgagttggtaatatagacacaaataaatatatatatttttatagcttaaaagtgtgttatgaatattttaagcatattttctttttctttggattgtgtaaaatgaactcatataatgcgtttttttgtttttgttattttgtcattttgttattttgctattttatggattaatttttgtttataaaatgggaaataattttaacatatttaaataaatggagaaaaaatataaaataattataaaaaaaagttaatacacattttttcttgttatagaccttatatttatttatccatatatatatatatatatatatatatatatatatacataccaagtgaattaagaggaaagctaatttattattcagaataatatatgaactatatataatttttattattttggtgtatattaatctgtctatatgcatacatgcaataatttatcgacttatatatcaaataacataaaatagaagtgttttaaattatggatatatgctcaatattcattttttttaataagttagctatatttaaattatacattttatatatggtctcttttttttttaaatattatttaagtgatcatgaaaatataaataatttttttttatttaatatacttttgcttgcatgtggtaaatggaaatttggatgtgttttgaaagttcgtatatagttgtatggacatataaatatattttgtgaaaaaattgtttttatgttttatactttatgccaatactttttgagtaaaacaaagcaagtgcttataaataattaaagccaattttataatatatatttttttatttaatttgaatttagtagtataattttttatggtaagtgctcaaagagagttgcttataaagtatggtttgtttctttttcgccattttgaattacacattaaaaatatatagatacatatattataatatgaaatcattaataatttagggaaattctacaaatttaaaaacgaataaaataattgtttttcattatgccataacacaatattgatatatacatgtacaaacatttttttttatttggaaaatataaattatataaaaaaaaatgtatagtatacaaaatgagcatattcacacggggtggacgttcattttttcatttttcccctgttttttatgagtatatgataaaattttatgaacatttacacaaaatgaaaatggatatataggaaaaatggagcggtatttcatttatctttgattgtcatttggatattatattaccttgggtaggcaattaaaaatgttaaataacaatttaaggaaattatattttatatattaaaattaacactgtattatatgattcgcttataaaagccactcttccccatgcaaagctgtttaatatcaattttaacaaatacacacatgttaatatatttatatatataatttatatatttataatttatatatttatatttttattatttatatatttattatttattgtgtgtgtcaattcgggtaggatatacctcttttttattgtttaaagcgatttgtattctaaaatataaagaatttgaaaaagagaaagatagaatatgatcccatcatatatagctctataatttttatttagcgcgaattaatttttctattaagtttatgtgtaattaaaataacggaatatatataatacaataaaaaagtgcataaattaaaattttttcaattaaatttttttttttaaggggttatataatattaaatatataaaatacgattatatatttttgctacaattttttatattaagatataaatagtaaataaatggtattatatggcatgtaatatataaattttttccaatttttattttatatacacttttccttttttttgtcataaaacttaaacaatttacacattcattttaaaaattgactatttgtttcaacattttttgagttttcgttttataatagtattttcatttgtatattgcttatatatatataaatacacacctaaatgttacaaaatggaa

SEQ ID NO: 3 - DNA, 1601 bp, Homo sapiens
ccttttctcagtttcattgaccaggctatctaacatttacgattcccccagcatatcatccattttattcttctttacggactcatttctggctcgtagtaaatgttcaataacggttaagtgaactgaatctctaatttctggtcgttaatttgtagagaccactttcccggatttaaaaagtgcaggtgctctggcagccttaagtcggtgaaaacaagaatattccgcaaaacaaaaataacttgtttattcatcaaaaacagttaacgttacacttctataccgtcaattcgtgtgtgaacagagttgtgtctgatccaagaccttacagttcaagcactggatcccaataacctgtaaacaacgttttgttatttgcaatgcaatcctgggctaacttttcaaaagtcgtttttctcaatttcccgttagttaaaggaataaaaaaatgctgtccaacgtggtcaatggaccaaacaaacaaggacaacaaacagaagcgtgagccattgctaacacaacagcgaccaaacaatcatgcactgtatcaggcgtgtaaaaatctggggggaaaggcttagtcttaaggcctaagtcatcggacactggtcctctcccggggaataaaacggcctcctcctctgagagggaaatgtcctgaattttccctgcagagggaaggtcacctgcaagcctacattcgtgacagcgttcccgaggaccccctgatcctcatcttcttttctgcttcatcagaccagagaccgtggctaaaagccccccatctgccggctctcaggggcttccgaggcggcggggcgggaggcggcgcccagtcaaggacaacagaagactacgtcgcgtgggccaaaggaaacagtcccgtcaataaataagcctctggccgacgcacctcacccttcccatccatacaagacctcaccttctccccggagccgcggaggctgttctgctaaactgtctggaccacgacgaccccctaggaggccgggtcgcttattcaatatggcggcctcggctaactctgtcagccgggcctggagaacggaaagcccggagggactagaatccttgcattcgacaagtttgtcggcaatttccgtcagccgaccagagggcggagctgaggtcggcccagcccggaggcgggaaggacaacttggacccgcagttccgccggaagtggccccagcctcgaggccgggcgtcttcggtcatctccggcgcttctagggctggttcccgtcatcttcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacaccatgggtttcggagacc

SEQ ID NO: 4 - DNA, 820 bp, Babesia microti
ccttctccactgctactttttagagagtattcattaccatgccagaaattaattgtaaaaagtacctcttatcaatgattatagcgtttaaattgctaaaattattttaatgatcaactaatcaagattcaacctataattcatatgttgacataaaagtatccctgatatgaatatatcataactgcatttgtaaacttgataattggaaaagaggataacctatcactaatatataaactttcataactacatcaattcattttttcgatgaagatagcagtgttaagataaaaatctatccattagtatataactgcaaaaacaattttattgccgttgctatcattaactataaagttataatgattaaaatatattatgtaaaaagatatgtgatacattcctttgtgattatttatgattgtaaattcatgtgtacgggaattaatttcatatttgatgttttgctaattcattaaatgactaaaattgacttatatttatattaaaatctagattatatattataaaattgatcgtctacatagattcataaatatatgagcaaattgcagaaatgtcaggtaattagtattaatttgttatcctttaattgccaaatgtaaattgttacttctccatcaaacagcatttctaacagttgttattttccgctactcccagattcactttcaatgttactgataactatataacatttgacatagttattatatcaatttaatcatttatttagtttattttcaaaatattttgacataaaattcatagatttatttctatgtccgaatatgat

SEQ ID NO: 5 - DNA, 716 bp, Babesia microti
agtaagtgctaaatgacgatttcgtataattaaatatattttaatgatgtaaaactagattcataatctaagtattatatcatatcacatgattccgcgccttaaaaagtatattaatatattaaaccaatgtgactttcaatccaacaaatttattgtataataatgactactacatgaaaagatgttattcgactaggtttgatatatttataatattaggtacatgttgtaacaataaattaattgaaaaacatcgtaatacaaataaagattgttcgtacatgtacattgtgacagtttaatctgcaatgttattaattattaccttatactgaaatttaactaattcatatacttaaaaacgatctatatggactaatatatatttgcgtaaaaatgaattgtcactagttactatatattctagttaatctactttagagaccttccaatgtcattctcgatagttatttacaataaaacatacatattgtgatttttaaaatatttagaatctttttatattcttatctttattattcacaagttggatatttcatccaaatcatttattgaatctggattattgtatgaaatcgtgcaaaagagcacaattataccccatctcctttataattttaaagctggttacttcaattcgtagtttgatcatttcttttttttaatttggagcatagaagtatgtggatttg

SEQ ID NO: 6 - DNA, 2958 bp, Artificial Sequence
  Plasmid/cloning vector
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 7 - DNA, 200 bp, Babesia microti
ttcattaaatatttaatagtcagtgttattcaaagtaactatgtgatagacattttaatatattaaatatgcagaactgaaaacatacattcataattagtactaagaatatctgtacaatatatatatatatatatatgcgcacaatatgtaaaatagttgctattaaatatatgattacatgctatatgtgtgtttga

SEQ ID NO: 8 - DNA, 30 bp, Artificial Sequence
  Primer
attctcgagtgccattgttattcacgctag

SEQ ID NO: 9 - DNA, 32 bp, Artificial Sequence
  Primer
gccaagcttttttctaacattcaagaggctgc

SEQ ID NO: 10 - DNA, 33 bp, Artificial Sequence
  Primer
gccaagcttatgagtaaaggagaagaacttttc

SEQ ID NO: 11 - DNA, 32 bp, Artificial Sequence
  Primer
attggatccttatttgtatagttcatccatgc

SEQ ID NO: 12 - DNA, 31 bp, Artificial Sequence
  Primer
attggatccacatgacacatacttggtgcgt

SEQ ID NO: 13 - DNA, 37 bp, Artificial Sequence
  Primer
attgcggccgcaccatgtcacaatacaacatattatg

SEQ ID NO: 14 - DNA, 33 bp, Artificial Sequence
  Primer
cggtctagaatggtgagcaagggcgaggaggat

SEQ ID NO: 15 - DNA, 33 bp, Artificial Sequence
  Primer
attcccgggctacttgtacagctcgtccatgcc

SEQ ID NO: 16 - DNA, 33 bp, Artificial Sequence
  Primer
attcccggggtatatcggggagagcggcaacgt

SEQ ID NO: 17 - DNA, 32 bp, Artificial Sequence
  Primer
attcccggggctacaagctggtgtgctttacc

SEQ ID NO: 18 - DNA, 33 bp, Artificial Sequence
  Primer
attctcgagatggtgagcaagggcgaggaggat

SEQ ID NO: 19 - DNA, 32 bp, Artificial Sequence
  Primer
attctcgaggctacaagctggtgtgctttacc

SEQ ID NO: 20 - DNA, 32 bp, Artificial Sequence
  Primer
attctcgagaagtgtgttatgaatattttaag

SEQ ID NO: 21 - DNA, 29 bp, Artificial Sequence
  Primer
gccgaattctttgtaacatttaggtgtgt

SEQ ID NO: 22 - DNA, 33 bp, Artificial Sequence
  Primer
gccgaattcatgagtaaaggagaagaacttttc

SEQ ID NO: 23 - DNA, 31 bp, Artificial Sequence
  Primer
attggatcctatttgtatagttcatccatgc

SEQ ID NO: 24 - DNA, 32 bp, Artificial Sequence
  Primer
attggtaccccttctccactgctactttttag

SEQ ID NO: 25 - DNA, 32 bp, Artificial Sequence
  Primer
attctcgagatcatattcggacatagaaataa

SEQ ID NO: 26 - DNA, 34 bp, Artificial Sequence
  Primer
attgcggccgcagtaagtgctaaatgacgatttc

SEQ ID NO: 27 - DNA, 32 bp, Artificial Sequence
  Primer
attgagctccaaatccacatacttctatgctc

SEQ ID NO: 28 - DNA, 27 bp, Artificial Sequence
  Primer
attctttaaatgcataaataataatag

SEQ ID NO: 29 - DNA, 25 bp, Artificial Sequence
  Primer
ccaatatatgccgaacaagctacag

SEQ ID NO: 30 - DNA, 22 bp, Artificial Sequence
  Primer
gaccacatggtccttcttgagt

SEQ ID NO: 31 - DNA, 26 bp, Artificial Sequence
  Primer
actgtatattatatggaattattcta

SEQ ID NO: 32 - DNA, 717 bp, Artificial Sequence
  Reporter Gene
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 33 - DNA, 711 bp, Artificial Sequence
  Reporter gene
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 34 - DNA, 1650 bp, Artificial Sequence
  Reporter gene
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtg

SEQ ID NO: 35 - DNA, 627 bp, Babesia microti
acatgacacatacttggtgcgttttgctacacaaatttccaacgcatctaattttcacaccaaaataatcgccatgctatgttttagtctattaattatgacttagattataaattttttcttaaatccccacaacgaatggccagtttcacggagactgtgcttgtattgtataagtcttatggacagatacggaatattcgcaggtaagtaacgtatattttacaatttgctatagaataaatgcttgttgaatatagaaactagactaaaatctagtctaggtgtgacacagtgtatttctaaccaataatttacatctgctattatgcgtgaagtatctcataacacaatgtacagttgaatttgactgtaaattttgggcttgcatgtgatattttaacgtttatatcctgtagaactgtagcttgttcggcatatattggccattcaacgtctaattataaatcttgtctactatgtgaatgcacctagtatccaaaatgtatggatggaatctaaacaatactaatactatacactactagcttctataaatcataccatatgctgtatctacagtgtagttatacatagatgacataatatgttgtattgtgacatggt